(19) United States
(12) Patent Application Publication
(10) Pub. No.: US 2005/0096288 A1
(43) Pub. Date: May 5, 2005

(54) Lipoproteins as nucleic acid vectors

(75) Inventor: Juan G. Guevara JR., Brownsville, TX (US)

Correspondence Address: Edwin Flores;Chalker Flores, LLP, 12700 Park Central Drive, Suite 455, Dallas, TX, 75251, US

(73) Assignee: Aragene, Inc., Houston, TX (US)

(21) Appl. No.: 10/895,250

(22) Filed: Jul. 20, 2004

Related U.S. Application Data
(63) Continuation-in-part of application No. 08/874,807, filed on Jun. 13, 1997, now abandoned.
(63) Continuation-in-part of application No. 09/079,030, filed on May 14, 1998, now Pat. No. 6,635,623.
(63) Continuation-in-part of application No. 10/656,053, filed on Sep. 5, 2003.

(51) Int. Cl. A61K 48/00; A61K 38/17; C12N 15/85

(52) U.S. Cl. 514/44; 514/21; 435/455

(57) ABSTRACT

The present invention relates to a composition and method for activating an antigen specific immune response using by providing a host with a native low density lipoprotein and a nucleic acid that expressed an antigen bound to the low density lipoprotein.

### TECHNICAL FIELD OF THE INVENTION

The present invention relates to materials and methods for vaccination using in vivo deliver of nucleic acids and, more particularly, to the use of lipoproteins, e.g., low density lipoproteins (“LDL”) and/or apolipoproteins, to deliver isolated and purified nucleic acids that express one or more antigens.

### BACKGROUND OF THE INVENTION

The present application is a continuation-in-part, and claims priority to, U.S. patent application Ser. No. 08/874,807, entitled “Lipoproteins As Nucleic Acid Vectors” filed Jun. 13, 1997, now abandoned and U.S. patent application Ser. No. 09/079,030, now U.S. Pat. No. 6,635,623, issued Oct. 21, 2003 and U.S. patent application Ser. No. 10/656,053, filed Sep. 5, 2003. The entire text of these disclosures is specifically incorporated by reference herein without disclaimer.

Many vaccines used currently are composed of live/attenuated pathogens that, when inoculated, infect cells and elicit a broad immune response in the host. Live vaccines are often superior to antigen or subunit vaccines because they tend to elicit a broad level protective response. However, serious disadvantages in using such vaccines include: the risk of a vaccine-induced infection; problems with producing and storing the vaccine; and failure to trigger any immune response. The failure to trigger an immune response is a particular challenge with cancer related antigens, as the cancer related antigen must be delivered to a cell that provides antigen processing, antigen presentation and co-stimulation of the T cell.

Pathogen vaccines generally generate antibodies to single proteins or to a limited number of proteins associated only with the pathogen. However, there is no assurance that antibodies produced in response to an antigen will provide protection against the pathogen providing the antigen. Ultimately, no single antigen may prove effective as a vaccine because the ability of subunit or killed vaccine preparations to elicit a broad immune response is generally quite limited.

Certain disadvantages of conventional vaccines are overcome by using so-called “genetic immunization.” Genetic immunization involves inoculating simple, naked plasmid DNA encoding a pathogen protein into the cells of the host. The pathogen's antigens are produced intracellularly and, depending on the attached targeting signals, can be directed toward, e.g., major histocompatibility complex (MHC) class I or II presentation. Using genetic immunization the risk of infection may be eliminated because only one or a few genes of the pathogen are delivered. While the production of genetic vaccines is straightforward because DNA is considerably more stable than proteinaceous or live/attenuated vaccines, its use is limited by, e.g., the degradation of the DNA during attempted delivery due to the presence of nucleases in hosts and host cells that degrade the DNA. However, despite promising initial results with genetic vaccination, there remains the more basic and unsolved problem of delivering the particular gene or genes of the pathogen that will express an immunogen capable of priming the immune system for rapid and protective response to pathogen challenge.

One such gene-vaccine system is disclosed in U.S. Pat. No. 6,410,241, issued to Sykes, et al., which teaches methods of screening open reading frames to determine whether they encode polypeptides with an ability to generate an immune response. The open reading frames that generate immune responses include linear expression elements (LEEs) and circular expression elements (CEEs), which are useful in a variety of molecular biology protocols. Specifically, the invention relates to the use of LEEs and CEEs to screen for gene function, biological effects of gene function, antigens, and promoter function. Also disclosed are methods of producing proteins, antibodies, antigens, and vaccines. Also, the invention relates to methods of making LEEs and CEEs, and LEEs and CEEs produced by such methods.

Gene delivery systems that use the viral entry mechanism of recombinant viral vectors have major disadvantages. Systems that use replication-defective adenoviral vectors can infect a wide variety of eukaryotic cell types including quiescent somatic cells utilizing the viral entry mechanism. However, adenoviral vector-based delivery systems are only successful in transient gene expression and repeated administration of the viral vector results in a strong immunological response of the host. In addition, the host will experience an adenoviral infection and can experience its symptoms if the recombinant vector undergoes homologous recombination with the wild-type virus strain. Systems that employ recombinant retroviral vectors can be used for stable integration of the gene of interest into the host's genome, but only actively dividing cells can be targeted. In addition, the disadvantages of the adenoviral vector systems also apply to retroviral vector systems, e.g., the development of an immune response to the delivery system and diseases associated with the vectors, genes delivered, promoter systems and the like.

### SUMMARY OF THE INVENTION

The present invention relates to a gene delivery system for use in vaccine therapy. More particularly, the present invention concerns the use of lipoproteins, including but not limited to, low density lipoproteins (“LDL”), and/or apolipoproteins for the in vivo transport of nucleic acids. In one embodiment, the present invention provides delivering a nucleic acid that encodes an antigen with an isolated polypeptide that includes at least one LDL or VLDL nucleic acid binding domain, wherein the nucleic acid is bound to the polypeptide portion of the LDL or VLDL. The LDL or VLDL nucleic acid binding domain binds specifically to the nucleic acid and is used to deliver, as taught herein, the gene for expression within a host cell.

The present invention also relates to compositions and methods for activation of the immune response, e.g., to prevent or treat a number of pathological states such as viral diseases and cancer through immunotherapy. Specific immunity requires two basic components: an antigen and an immune response mechanism that responds specifically to the presence of the antigen. For centuries specific immunity has been achieved using vaccination with antigens, e.g., portions of a pathogen, live/attenuated pathogens and the like. One particular advantage of the present invention is that it permits, for the first time, the specific delivery of an antigen encoding gene with a high efficiency to the site of processing and presentation. The efficient delivery of the antigen encoding gene to a host cell permits the host cell to efficiently process the antigen for loading onto protein of the Class I or Class II Major Histocompatibility Complex (MHC) using native antigen processing enzymes. In another embodiment, the nucleic acid may include the expression region operably linked to a cognate promoter or a native promoter active in, e.g., eukaryotic cells. Generally, the expression region may encode a portion or the complete antigenic polypeptide, however, the antigen may be provided as a concatamer or be provided in multiple copies with linker regions that are processed in the lumen of the endoplasmic reticulum for presentation by class I or class II MHC.

Examples of antigens for the LDL vaccine of the present invention include antigens such as genes expressed in certain cancers (e.g., MAGE, GAGE, BAGE, DAGE and the like), allergies, auto-immune disease and infectious diseases (fungal, bacterial, viral, helminthic, etc.). The expression region may be linked to a promoter selected from, e.g., CMV IE, LTR, SV40 IE, HSV tk, β-actin, human globin α, human globin β and human globin γ promoter. The nucleic acid binding domain may be an apoB100, apoA1, apoA-II, apoA-IV, acat, apoE, apoC-II, apoC-III and/or apo-D nucleic acid binding domain. The nucleic acid binding domain and/or the complete apoB100 protein may be, e.g., apoB100 from human, rat and baboon low density apoB100 and the like.

In another embodiment, the nucleic acid binding domain of LDL or VLDL may further include at least one nuclear localization sequence. More particularly, the nuclear localization sequence may be from apoB100. Examples of the nuclear localization regions of LDL, VLDL or other proteins are disclosed in U.S. Pat. No. 6,635,623, relevant portions incorporated herein by reference.

A method of the present invention includes expressing an antigenic polypeptide in a human cell by providing a composition that includes: (i) an isolated polypeptide with at least one LDL or VLDL nucleic acid binding domain and (ii) a nucleic acid that includes an expression cassette encoding an antigenic polypeptide or an open reading frame from a pathogen and a promoter active in eukaryotic cells, wherein the coding sequence is linked operably to the promoter, and wherein the nucleic acid sequence is bound to the LDL or VLDL; contacting the composition with the cell under conditions permitting transfer of the composition into the cell; and culturing the cell under conditions permitting the expression of the polypeptide.

The present invention also includes a method for providing an expression construct to a human cell by providing a composition that includes: (i) an isolated polypeptide that includes at least one LDL or VLDL nucleic acid binding domain and (ii) an expression cassette including a nucleic acid sequence encoding at least a portion of an antigen, a chimera, a fusion protein or a concatamer of an antigen and a promoter active in eukaryotic cells, wherein the expression region is operably linked to the promoter, and wherein the nucleic acid sequence is bound to the LDL or VLDL; contacting the composition with the cell under conditions permitting transfer of the composition into the cell; and culturing the cell under conditions permitting the expression of the antigen.

Further the present invention contemplates a method for treating a human disease by providing a composition that includes: (i) an isolated polypeptide including at least one LDL or VLDL nucleic acid binding domain and (ii) an antigen expression cassette, wherein the antigen expression cassette is bound to the LDL or VLDL; and administering the composition to a human subject having a disease that may be treated with a vaccine for the antigen under conditions permitting transfer of the composition into cells of the human subject.

In specific embodiments, the disease may be, e.g., cancer, allergies, auto-immune disease and infectious diseases. In one embodiment the nucleic acid encoding the antigen includes one or more LDL or VLDL nucleic acid binding sequences, whether native to the sequence or added in “cis” or “in trans” with the nucleic acid encoding the antigen. By the sequence being in “cis,” it is meant that the nucleic acid is contiguous with the antigen encoding nucleic acid, in contrast by “in trans” it is meant that the nucleic acid is attached to the nucleic acid encoding the antigen by a covalent or non-covalent attachment that is other than a 5′ to 3′ phosphate link, e.g., by attaching to the base or with a bivalent cross-linker.

Yet another embodiment of the present invention is a pharmaceutical composition that includes at least one LDL or VLDL nucleic acid binding domain; and an isolated and purified, antigen-encoding nucleic acid having one or more LDL or VLDL nucleic acid binding domain-binding sequence, wherein the nucleic acid is bound to the LDL or VLDL nucleic acid binding domain; the pharmaceutical composition being dispersed in a suitable diluent.

Also contemplated by the present invention is a method of transforming or transfecting a cell by providing a cell; contacting the cell with a composition that includes: (i) an isolated LDL or VLDL nucleic acid binding domain polypeptide, and (ii) an expression cassette that expressed an antigen wherein the nucleic acid sequence is bound to the LDL or VLDL; and expression of the antigen is indicative of the transformation or transfection.

### BRIEF DESCRIPTION OF THE DRAWINGS

For a more complete understanding of the features and advantages of the present invention, reference is now made to the detailed description of the invention along with the accompanying figures and in which:

FIGS. 1-8 show an amino acid sequence (single letter code) alignment of the DNA-binding domains to a region in apo b for 8 different proteins (see below) (SEQ ID NOS.: 1-8, respectively); more particularly:

FIG. 1 shows the location of P (proline) Motifs;

FIG. 2 is an alignment of P Motifs;

FIG. 3 shows location of K (lysine) and R (arginine), positively charged amino acids;

FIG. 4 shows an alignment of Positively Charged Amino Acids, K (lysine) and R (arginine);

FIG. 5 shows an alignment of Negatively Charge Residues, E (glutamic acid) and D (asparatic acid);

FIG. 6 shows an alignment of Polar Residues, S (serine), T (threonine), Q (glutamine), N (asparagine), Y (tyrosine), H (histidine), C (cysteine), and W (tryptophan);

FIG. 7 shows an alignment of Non-polar Residues, A (alanine), V (valine), L (leucine), I (isoleucine), M (methionine), and F (phenylalanine);

FIG. 8 shows the location of G (glycine) Residues; and

FIG. 9 is a generalized construct for use with the present invention.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

The present invention arises from the discovery that regions of apolipoproteins, the protein fraction of lipoprotein particles, are similar in primary structure and amino acid sequence to cellular proteins which are known to bind to DNA. Presently, the only known functions of lipoproteins VLDL, IDL, LDL and HDL are the solubilization and transport of hydrophobic lipids in plasma. The instant invention shows that LDLs, but not other lipoproteins, form a complex with DNA.

Herein, synthetic analogues of regions of DNA have been shown to bind to highly purified preparations of human, rat, and baboon LDL but not to other human lipoproteins such as VLDL and HDL, nor to mouse lipoproteins. In fact, the differences observed among the four species tested suggest that human, rat, and baboon lipoproteins behave very similarly in terms of DNA binding preference. Further, purified preparations of human, rat, and baboon LDLs are shown to complex with the promoter region of the human cytomegalovirus. Thus, the present invention demonstrates that human LDL complexes with specific regions of genomic DNA.

The term “antigen” as used herein refers to a molecule that can initiate a humoral and/or cellular immune response in a recipient of the antigen. The antigen is usually an agent that causes a disease for which a vaccination would be advantageous treatment. When the antigen is presented on MHC, the peptide is often about 8 to about 25 amino acids. Antigens include any type of biologic molecule, including, for example, simple intermediary metabolites, sugars, lipids and hormones as well as macromolecules such as complex carbohydrates, phospholipids, nucleic acids and proteins. Common categories of antigens include, but are not limited to, viral antigens, bacterial antigens, fungal antigens, protozoal and other parasitic antigens, tumor antigens, antigens involved in autoimmune disease, allergy and graft rejection, and other miscellaneous antigens.

Examples of viral antigens include, but are not limited to, e.g., retroviral antigens such as retroviral antigens from the human immunodeficiency virus (HIV) antigens such as gene products of the gag, pol, and env genes, the Nef protein, reverse transcriptase, and other HIV components; hepatitis viral antigens such as the S, M, and L proteins of hepatitis B virus, the pre-S antigen of hepatitis B virus, and other hepatitis, e.g., hepatitis A, B, and C, viral components such as hepatitis C viral RNA; influenza viral antigens such as hemagglutinin and neuraminidase and other influenza viral components; measles viral antigens such as the measles virus fusion protein and other measles virus components; rubella viral antigens such as proteins E1 and E2 and other rubella virus components; rotaviral antigens such as VP7sc and other rotaviral components; cytomegaloviral antigens such as envelope glycoprotein B and other cytomegaloviral antigen components; respiratory syncytial viral antigens such as the RSV fusion protein, the M2 protein and other respiratory syncytial viral antigen components; herpes simplex viral antigens such as immediate early proteins, glycoprotein D, and other herpes simplex viral antigen components; varicella zoster viral antigens such as gpI, gpII, and other varicella zoster viral antigen components; Japanese encephalitis viral antigens such as proteins E, M-E, M-E-NS1, NS1, NS1-NS2A, 80% E, and other Japanese encephalitis viral antigen components; rabies viral antigens such as rabies glycoprotein, rabies nucleoprotein and other rabies viral antigen components. See Fundamental Virology, Second Edition, eds. Fields, B. N. and Knipe, D. M. (Raven Press, New York, 1991) for additional examples of viral antigens.

Bacterial antigens for use with the LDL Vaccine disclosed herein include, but are not limited to, e.g., bacterial antigens such as pertussis toxin, filamentous hemagglutinin, pertactin, FIM2, FIM3, adenylate cyclase and other pertussis bacterial antigen components; diptheria bacterial antigens such as diptheria toxin or toxoid and other diptheria bacterial antigen components; tetanus bacterial antigens such as tetanus toxin or toxoid and other tetanus bacterial antigen components; streptococcal bacterial antigens such as M proteins and other streptococcal bacterial antigen components; gram-negative bacilli bacterial antigens such as lipopolysaccharides and other gram-negative bacterial antigen components, Mycobacterium tuberculosis bacterial antigens such as mycolic acid, heat shock protein 65 (HSP65), the 30 kDa major secreted protein, antigen 85A and other mycobacterial antigen components; Helicobacter pylori bacterial antigen components; pneumococcal bacterial antigens such as pneumolysin, pneumococcal capsular polysaccharides and other pneumococcal bacterial antigen components; haemophilus influenza bacterial antigens such as capsular polysaccharides and other haemophilus influenza bacterial antigen components; anthrax bacterial antigens such as anthrax protective antigen and other anthrax bacterial antigen components; rickettsiae bacterial antigens such as rompA and other rickettsiae bacterial antigen component. Also included with the bacterial antigens described herein are any other bacterial, mycobacterial, mycoplasmal, rickettsial, or chlamydial antigens.

Fungal antigens for use with compositions and methods of the invention include, but are not limited to, e.g., candida fungal antigen components; histoplasma fungal antigens such as heat shock protein 60 (HSP60) and other histoplasma fungal antigen components; cryptococcal fungal antigens such as capsular polysaccharides and other cryptococcal fungal antigen components; coccidiodes fungal antigens such as spherule antigens and other coccidiodes fungal antigen components; and tinea fungal antigens such as trichophytin and other coccidiodes fungal antigen components.

Examples of protozoal and other parasitic antigens include, but are not limited to, e.g., plasmodium falciparum antigens such as merozoite surface antigens, sporozoite surface antigens, circumsporozoite antigens, gametocyte/gamete surface antigens, blood-stage antigen pf 155/RESA and other plasmodial antigen components; toxoplasma antigens such as SAG-1, p30 and other toxoplasmal antigen components; schistosomae antigens such as glutathione-S-transferase, paramyosin, and other schistosomal antigen components; leishmania major and other leishmaniae antigens such as gp63, lipophosphoglycan and its associated protein and other leishmanial antigen components; and trypanosoma cruzi antigens such as the 75-77 kDa antigen, the 56 kDa antigen and other trypanosomal antigen components.

Tumor antigens include, but are not limited to, e.g., telomerase; multidrug resistance proteins such as P-glycoprotein; DAGE, GAGE, BAGE, MAGE-1, 2, 3 and the like, alpha fetoprotein, carcinoembryonic antigen, mutant p53, papillomavirus antigens, gangliosides or other carbohydrate-containing components of melanoma or other tumor cells. It is contemplated by the invention that antigens from any type of tumor cell can be used in the compositions and methods described herein. Examples of other miscellaneous antigens involved in one or more types of autoimmune response include, e.g., endogenous hormones such as luteinizing hormone, follicular stimulating hormone, testosterone, growth hormone, prolactin, and other hormones.

Antigens involved in autoimmune diseases, allergy, and graft rejection can be used in the compositions and methods of the invention. For example, an antigen involved in any one or more of the following autoimmune diseases or disorders can be used in the present invention: diabetes mellitus, arthritis (including rheumatoid arthritis, juvenile rheumatoid arthritis, osteoarthritis, psoriatic arthritis), multiple sclerosis, myasthenia gravis, systemic lupus erythematosis, autoimmune thyroiditis, dermatitis (including atopic dermatitis and eczematous dermatitis), psoriasis, Sjogren's Syndrome, including keratoconjunctivitis sicca secondary to Sjogren's Syndrome, alopecia areata, allergic responses due to arthropod bite reactions, Crohn's disease, aphthous ulcer, iritis, conjunctivitis, keratoconjunctivitis, ulcerative colitis, asthma, allergic asthma, cutaneous lupus erythematosus, scleroderma, vaginitis, proctitis, drug eruptions, leprosy reversal reactions, erythema nodosum leprosum, autoimmune uveitis, allergic encephalomyelitis, acute necrotizing hemorrhagic encephalopathy, idiopathic bilateral progressive sensorineural hearing loss, aplastic anemia, pure red cell anemia, idiopathic thrombocytopenia, polychondritis, Wegener's granulomatosis, chronic active hepatitis, Stevens-Johnson syndrome, idiopathic sprue, lichen planus, Crohn's disease, Graves ophthalmopathy, sarcoidosis, primary biliary cirrhosis, uveitis posterior, and interstitial lung fibrosis. Examples of antigens involved in autoimmune disease include glutamic acid decarboxylase 65 (GAD 65), native DNA, myelin basic protein, myelin proteolipid protein, acetylcholine receptor components, thyroglobulin, and the thyroid stimulating hormone (TSH) receptor. Examples of antigens involved in allergy include pollen antigens such as Japanese cedar pollen antigens, ragweed pollen antigens, rye grass pollen antigens, animal derived antigens such as dust mite antigens and feline antigens, histocompatiblity antigens, and penicillin and other therapeutic drugs. Examples of antigens involved in graft rejection include antigenic components of the graft to be transplanted into the graft recipient such as heart, lung, liver, pancreas, kidney, and neural graft components. The antigen may be an altered peptide ligand useful in treating an autoimmune disease.

As used herein, the term “epitope(s)” refer to a peptide or protein antigen that includes a primary, secondary or tertiary structure similar to an epitope located within any of a number of pathogen polypeptides encoded by the pathogen DNA or RNA. The level of similarity will generally be to such a degree that monoclonal or polyclonal antibodies directed against such polypeptides will also bind to, react with, or otherwise recognize, the peptide or protein antigen. Various immunoassay methods may be employed in conjunction with such antibodies, such as, for example, Western blotting, ELISA, RIA, and the like, all of which are known to those of skill in the art. The identification of pathogen epitopes, and/or their functional equivalents, suitable for use in vaccines is part of the present invention. Once isolated and identified, one may readily obtain functional equivalents. For example, one may employ the methods of Hopp, as taught in U.S. Pat. No. 4,554,101, incorporated herein by reference, which teaches the identification and preparation of epitopes from amino acid sequences on the basis of hydrophilicity. The methods described in several other papers, and software programs based thereon, can also be used to identify epitopic core sequences (see, for example, Jameson and Wolf, 1988; Wolf et al., 1988; U.S. Pat. No. 4,554,101). The amino acid sequence of these “epitopic core sequences” may then be readily incorporated into peptides, either through the application of peptide synthesis or recombinant technology.

As used herein, the term “promoter” describes a control sequence that is a region of a nucleic acid sequence at which initiation and rate of transcription are controlled. It may contain genetic elements at which regulatory proteins and molecules may bind such as RNA polymerase and other transcription factors. The phrases “operatively positioned,” “operatively linked,” “under control,” and “under transcriptional control” mean that a promoter is in a correct functional location and/or orientation in relation to a nucleic acid sequence (i.e., ORF) to control transcriptional initiation and/or expression of that sequence. A promoter may or may not be used in conjunction with an “enhancer,” which refers to a cis-acting regulatory sequence involved in the transcriptional activation of a nucleic acid sequence. A listing of promoters and/or enhancers that may be used with the present invention is described in, e.g., U.S. Pat. No. 6,410,241, relevant descriptions and tables incorporated herein by reference.

As used herein, the terms “cell,” “cell line,” and “cell culture” may be used interchangeably. All of these terms also include their progeny, which is any and all subsequent generations, in vivo, ex vivo or in vitro. It is understood that all progeny may not be identical due to deliberate or inadvertent mutations. In the context of expressing a heterologous nucleic acid sequence, “host cell” refers to a prokaryotic or eukaryotic cell, and it includes any transformable organism that is capable of expressing a heterologous gene encoded by a vector as delivered using the LDL protein vector of the present invention. A host cell can, and has been, used as a recipient for vectors. A host cell may be “transfected” or “transformed,” which refers to a process by which the exogenous nucleic acid expressing an antigen, as disclosed herein, is transferred or introduced into the host cell. A transformed cell includes the primary subject cell and its progeny.

The preparation of vaccine compositions that includes the nucleic acids that encode antigens of the invention as the active ingredient, may be prepared as injectables, either as liquid solutions or suspensions; solid forms suitable for solution in, or suspension in, liquid prior to infection can also be prepared. The preparation may be emulsified, encapsulated in liposomes. The active immunogenic ingredients are often mixed with carriers which are pharmaceutically acceptable and compatible with the active ingredient.

The term “pharmaceutically acceptable carrier” refers to a carrier that does not cause an allergic reaction or other untoward effect in subjects to whom it is administered. Suitable pharmaceutically acceptable carriers include, for example, one or more of water, saline, phosphate buffered saline, dextrose, glycerol, ethanol, or the like and combinations thereof. In addition, if desired, the vaccine can contain minor amounts of auxiliary substances such as wetting or emulsifying agents, pH buffering agents, and/or adjuvants which enhance the effectiveness of the vaccine. Examples of adjuvants that may be effective include but are not limited to: aluminum hydroxide, N-acetyl-muramyl-L-threonyl-D-isoglutamine (thr-MDP), N-acetyl-nor-muramyl-L-alanyl-D-isoglutamine, MTP-PE and RIBI, which contains three components extracted from bacteria, monophosporyl lipid A, trehalose dimycolate and cell wall skeleton (MPL+TDM+CWS) in a 2% squalene/Tween 80 emulsion. Other examples of adjuvants include DDA (dimethyldioctadecylammonium bromide), Freund's complete and incomplete adjuvants and QuilA. In addition, immune modulating substances such as lymphokines (e.g., IFN-γ, IL-2 and IL-12) or synthetic IFN-γ inducers such as poly I:C can be used in combination with adjuvants described herein.

Vaccine or treatment compositions of the invention may be administered parenterally, by injection, for example, either subcutaneously or intramuscularly. Additional formulations which are suitable for other modes of administration include suppositories, and in some cases, oral formulations or formulations suitable for distribution as aerosols. In the case of the oral formulations, the manipulation of T-cell subsets employing adjuvants, antigen packaging, or the addition of individual cytokines to various formulation that result in improved oral vaccines with optimized immune responses. For suppositories, traditional binders and carriers may include, for example, polyalkylene glycols or triglycerides; such suppositories may be formed from mixtures containing the active ingredient in the range of 0.5% to 10%, preferably 1%-2%. Oral formulations include such normally employed excipients as, for example, pharmaceutical grades of mannitol, lactose, starch magnesium stearate, sodium saccharine, cellulose, magnesium carbonate, and the like. These compositions take the form of solutions, suspensions, tablets, pills, capsules, sustained release formulations or powders and contain 10%-95% of active ingredient, preferably 25-70%.

The antigen encoding nucleic acids of the invention may be formulated into the vaccine or treatment compositions as neutral or salt forms. Pharmaceutically acceptable salts include the acid addition salts (formed with free amino groups of the peptide) and which are formed with inorganic acids such as, for example, hydrochloric or phosphoric acids, or with organic acids such as acetic, oxalic, tartaric, maleic, and the like. Salts formed with the free carboxyl groups can also be derived from inorganic bases such as, for example, sodium, potassium, ammonium, calcium, or ferric hydroides, and such organic bases as isopropylamine, trimethylamine, 2-ethylamino ethanol, histidine, procaine, and the like.

Vaccine or treatment compositions are administered in a manner compatible with the dosage formulation, and in such amount as will be prophylactically and/or therapeutically effective. The quantity to be administered depends on the subject to be treated, including, e.g., capacity of the subject's immune system to synthesize antibodies, and the degree of protection or treatment desired. Suitable dosage ranges are of the order of several hundred micrograms active ingredient per vaccination with a range from about 0.1 mg to 1000 mg, such as in the range from about 1 mg to 300 mg, and preferably in the range from about 10 mg to 50 mg. Suitable regiments for initial administration and booster shots are also variable but are typified by an initial administration followed by subsequent inoculations or other administrations. Precise amounts of active ingredient required to be administered depend on the judgment of the practitioner and may be peculiar to each subject. It will be apparent to those of skill in the art that the therapeutically effective amount of nucleic acid molecule or fusion polypeptides of this invention will depend, inter alia, upon the administration schedule, the unit dose of antigen administered, whether the nucleic acid molecule or fusion polypeptide is administered in combination with other therapeutic agents, the immune status and health of the recipient, and the therapeutic activity of the particular nucleic acid molecule or fusion polypeptide.

The compositions can be given in a single dose schedule or in a multiple dose schedule. A multiple dose schedule is one in which a primary course of vaccination may include, e.g., 1-10 separate doses, followed by other doses given at subsequent time intervals required to maintain and or reinforce the immune response, for example, at 1-4 months for a second dose, and if needed, a subsequent dose(s) after several months. Periodic boosters at intervals of 1-5 years, usually 3 years, are desirable to maintain the desired levels of protective immunity. The course of the immunization can be followed by in vitro proliferation assays of peripheral blood lymphocytes (PBLs) co-cultured with ESAT6 or ST-CF, and by measuring the levels of IFN-γ released from the primed lymphocytes. The assays may be performed using conventional labels, such as radionucleotides, enzymes, fluorescent labels and the like. These techniques are known to one skilled in the art and can be found in U.S. Pat. Nos. 3,791,932, 4,174,384 and 3,949,064, relevant portions incorporated by reference.

Because lipoproteins have specific cell membrane receptors and are actively and specifically internalized by many different cell types in mammals, and because the inventors show that LDL can bind DNA, these lipoproteins can be used as gene delivery vectors. More specifically, this invention relates to materials and methods for the use of lipoproteins, such as LDL, or, for example, apolipoproteins such as, but not limited to, apoB-100, apoA1, apoE, apoAIV, and apoC, or more specifically still, the DNA binding regions of these lipoproteins, as gene delivery vectors in vivo. As explained in greater detail below, the various embodiments of this invention include, but are not limited to, the delivery, of nucleic acids to a cell in the form of an LDL-lipoprotein complex, the specific delivery of DNA to the nucleus, and the specific localization of delivered DNA to specific nuclear sites.

Plasma levels of DNA increase in a variety of chronic diseases including lupus erythrematosis (Steinman, 1984), viral hepatitis (Neurath et al., 1984), and a variety of cancers (Leon et al., 1977; Shapiro et al., 1983; Stroun et al., 1987; Nawroz et al., 1996; Anker et al., 1997; Chen et al., 1996). It has also shown that lipoproteins in the blood of non-tumor carrying organisms are not bound to nucleic acids. However, cancer-carrying individuals, and in particular individuals with metastatic cancers, release large amounts of nucleic acids, into the blood. Thus, this invention also relates to the observation that lipoproteins in the blood of cancer patients and especially metastatic cancer patients are bound to nucleic acids, including DNA. Accordingly, this invention also may be used to provide a simple screening test for the presence or absence of cancer, especially metastatic cancer, by isolating a patient's lipoproteins and determining whether the lipoproteins are bound to nucleic acids; the presence of lipoprotein-bound nucleic acid being correlative with the presence of cancer and/or metastatic cancer in the living body. Further embodiments of the present invention relate to the sequence specific detection of DNA bound to lipoproteins in a cancer patient as a method for the identification of specific types of cancer in a living body. These and other aspects of the present invention are discussed in greater detail below.

Helper or cytotoxic T lymphocytes may be activated using antigen presenting cells (APCs) with an immunogenic peptide bound to selected major histocompatibility complex (MHC) molecules. Using cytotoxic T cells or CD8 cells as an example of T cell activation, it is known generally that these cells act as the main line of defense against viral infections. CTLs recognize specifically and kill cells that are infected. The T cell receptors on the surface of CTLs cannot recognize foreign antigens directly, but rather, recognize antigen presented to the T cell receptors by class I MHC for activation to occur. Conversely, T helper cells recognize antigen in the context of class II MHC on APCs.

The presentation of antigen to T cells is accomplished by the major histocompatibility complex (MHC) molecules. The major histocompatibility complex (MHC) is a large genetic locus encoding an extensive family of glycoproteins, which play an important role in the immune response. The MHC genes, which in humans are referred to as the HLA (human leukocyte antigen) complex, are located on human chromosome 6. Proteins encoded by MHC genes are present on cell surfaces and are largely responsible for recognition of tissue transplants as “non-self.”

MHC molecules are classified as either class I or class II molecules. Class I MHC molecules are expressed on almost all nucleated cells and are recognized by CTLs. T cells that act as helper cells express CD4 and are primarily restricted to Class II molecules, whereas CD8-expressing cells, represented by cytotoxic effector cells, interact with class I molecules. Class II MHC molecules are expressed primarily on cells involved in initiating and sustaining immune responses, such as T lymphocytes, B lymphocytes, macrophages, etc. Class II MHC molecules are recognized by helper T lymphocytes and induce proliferation of helper T lymphocytes and amplification of the immune response to the particular immunogenic peptide displayed on the class II MHC.

In order to present the antigen, the antigen must be either endogenously synthesized or endocytosed by the cell and a portion of the protein antigen degraded or processed into small peptide fragments in the endoplasmic reticulum and/or endosomes. Some of these small peptides are translocated into a pre-Golgi compartment and interact with, e.g., class I heavy chains to facilitate proper folding and association with the β2 microglobulin subunit of class I MHC. The mature peptide-MHC class I complex is routed to the cell surface for presentation and recognition by specific CTLs. MHC class I molecules present the peptide in the peptide binding groove created by the folding of the α1 and α2 domains of the class I heavy chain. Skilled immunologists will recognize that a similar chain of events transpires for class II antigen presentation of peptides on the α- and β-chains of class II.

LIPOPROTEINS. Lipoproteins appear as micro-pseudomicellar particles in the blood plasma of all mammalian species including humans. Their major function is to transport lipids and other hydrophobic compounds (i.e., fat-soluble vitamins) through the aqueous environment of the blood stream to their specific target cells. The transported lipids can be used as a major substrate for energy metabolism (i.e., triglycerides), structural components for cell membranes (i.e., phospholipids and cholesterol), or as precursors for steroid hormones and bile acids (i.e., cholesterol). Although, lipoproteins vary widely in size and lipid content, they have a common general structure. Lipoprotein particles are believed to be spherical and consist of a hydrophobic core containing nonpolar lipids surrounded by a hydrophilic surface monolayer of polar lipids and proteins, which are called apolipoproteins.

Plasma lipoproteins may be separated into five major classes based on their density, size, and compositional and functional properties: (1) chylomicrons, (2) very low density lipoproteins (VLDL), (3) intermediate lipoproteins (IDL), (4) low density lipoproteins (LDL), and (5) high density lipoproteins (HDL). The different classes of lipoproteins show distinct compositional differences in apolipoprotein content. The specific role of each class of lipoproteins in lipid metabolism is determined by the interaction of these apolipoproteins with specific enzymes and cellular receptors.

ApoB-100 Structure and Function. The major protein constituent of LDL is apoB-100. ApoB-100 is one of two known natural ligands for the LDL (apoE/apoB) receptor which is found on the surface of a wide variety of mammalian cell types (Brown and Goldstein, 1986). LDLs are taken up by a process called receptor-mediated endocytosis (Brown and Goldstein, 1986). Hence, lipoproteins may be able to function as naturally-occurring liposomes which contain protein constituents that can bind specifically to nucleic acids and can be internalized by a wide variety of eukaryotic cell types via specific receptor mediated processes.

Human apolipoprotein B-100 (apoB-100) is a major apoprotein component of very-low density lipoproteins (VLDL), intermediate density lipoproteins (IDL), low density lipoproteins (LDL), and lipoprotein[a] (Lp[a]). ApoB-100 is synthesized and incorporated into VLDL and Lp[a] by the liver. Human LDL can be described as a spherical particle composed of a hydrophobic core of cholesterol esters and triglycerides encapsulated by an amphipathic monolayer of phospholipids, glycolipids and cholesterol in which the apoB-100 is partially imbedded (Myant, 1990). In addition to one molecule of apoB-100, LDL is known to contain varying numbers of apo C-I, apo C-II, apo C-III, apo E, and apo D (Blanco-Vaca et al., 1992; Connelly et al., 1993; Blanco-Vaca et al., 1994).

The present invention may be used in conjunction with, e.g., the materials and methods described in U.S. Pat. No. 5,989,553, issued to Johnston, et al., entitled, Expression library immunization, which teaches a general method for vaccinating against any pathogen is presented. The method uses an expression library immunization, where an animal is inoculated with an expression library constructed from fragmented genomic DNA of the pathogen. All potential epitopes of the pathogen's proteins are encoded in its DNA, and genetic immunization is used to directly introduce one or more expression library clones to the immune system, producing an immune response to the encoded protein. Inoculation of expression libraries representing portions of the Mycoplasma pulmonis genome was shown to protect mice from subsequent challenge by this natural pathogen, e.g., protection against Listeria sp.

Other regions of the apoB-100 molecule are similar to specific regions in other known DNA binding proteins including, but not limited to ISGF3γ, coiled-coil regions of GCN4 and hMLKI, and the proline-pipe sequences of Tus. Further, the inventors found that the amino acid sequence of apolipoproteins, such as apoB-100 have regions involved with nucleotide binding and nuclear localization. For example, apolipoproteins such as apoB-100 show homology to the SH1 kinase domains of protein tyrosine kinases and the HIGH and KMSK motif plus critical lysine of tRNA synthetases both known to bind ATP as well as to the basic helix-loop-helix motif of sterol regulatory element binding proteins (SREBPs) known to localize to the nucleus where they are involved in the regulation of transcription.

Expression of apoB100. In certain embodiments of the present invention, it will be necessary to obtain apoB100 or lipoproteins containing apoB100 for use as DNA binding compositions. In particular embodiments as described herein below, such apoB100 may be obtained from the lipoprotein fraction of primate serum. As an alternative to purifying apoB100 from LDL fraction of serum, it is possible to generate pure fractions of apoB-100 by recombinant expression of the apoB100 gene. The apoB100 gene can be inserted into an appropriate expression system. The gene can be expressed in any number of different recombinant DNA expression systems to generate large amounts of the polypeptide product, which can then be purified and used as a DNA binding composition as described herein.

In one embodiment, specific amino acid sequence domains of an apoB100 polypeptide can be prepared. These may, for instance, be minor sequence variants of a polypeptide that arise due to natural variation within the population or they may be homologues found in other species. They also may be sequences that do not occur naturally but that are sufficiently similar that they function similarly and/or elicit an immune response that cross-reacts with natural forms of the polypeptide.

The nucleotide binding, nuclear localization and signal transduction domains of the apoB100 molecule are discussed in detail herein below. Recombinant technologies, well known to those of skill in the art, may be used to produce recombinant apoB100 with one or more of these domains having sequences that optimize the DNA binding and/or nuclear localization capacities of the molecule, decreased susceptibility to proteases and the use of conservative or partially-conservative amino acid substitutions. Furthermore, in certain instances it may be necessary to “customize” such domains in order to increase binding to a particular DNA sequence whilst decreasing the binding to other sequences as are known to the skilled artisan. Alternatively, it may be useful to alter a particular apoB100 polypeptide, in order to decrease its binding affinity for a particular molecule. Accordingly, sequence variants of these domains can be prepared by standard methods of site-directed mutagenesis such as those described below in the following section. Amino acid sequence variants of an apoB100 polypeptide, or particular domains therein can be substitutional, insertional or deletion variants. Deletion variants lack one or more residues of the native protein which are not essential for function or immunogenic activity.

Insertional variants include fusion proteins such as those used to allow rapid purification of the polypeptide and also can include hybrid proteins containing sequences from other proteins and polypeptides which are homologues of the polypeptide. For example, an insertional variant could include portions of the amino acid sequence of the polypeptide, from one species, together with portions of the homologous polypeptide from another species. Other insertional variants can include those in which additional amino acids are introduced within the coding sequence of the polypeptide. These typically are smaller insertions than the fusion proteins described above and are introduced, for example, into a protease cleavage site. Alternatively, insertional variants of the present invention may be created in which one or more DNA binding domains and nuclear localization domain have been added to a native apoB100 molecule to alter particular characteristics of the molecule.

In one embodiment, major antigenic determinants of the polypeptide are identified by an empirical approach in which portions of the gene encoding the polypeptide are expressed in a recombinant host, and the resulting proteins tested for their ability to elicit an immune response. Alternatively, the antigenic determinant may use Expressed Library Immunization (ELI) as taught by U.S. Pat. No. 6,410,241, relevant portions such as techniques, materials and methods incorporated herein by reference. For example, PCR can be used to prepare a range of cDNAs encoding peptides lacking successively longer fragments of the C-terminus of the protein. The immunoprotective activity of each of these peptides then identifies those fragments or domains of the polypeptide that are essential for this activity. Further studies in which only a small number of amino acids are removed at each iteration allows for the location of the antigenic determinants of the polypeptide.

Another embodiment for the preparation of polypeptides according to the invention is the use of peptide mimetics. Mimetics are peptide-containing molecules that mimic elements of protein secondary structure. See, for example, Johnson et al., “Peptide Turn Mimetics” in BIOTECHNOLOGY AND PHARMACY, Pezzuto, et al., Eds., Chapman and Hall, New York (1993). The underlying rationale behind the use of peptide mimetics is that the peptide backbone of proteins exists chiefly to orient amino acid side chains in such a way as to facilitate molecular interactions, such as those of antibody and antigen. A peptide mimetic is expected to permit molecular interactions similar to the natural molecule.

PURIFICATION OF LIPOPROTEINS. LDL isolation from plasma. One method for isolating LDL for use with the present invention begins by collecting fresh plasma, e.g., from a human. The plasma bag may include, e.g., sodium azide, sodium citrate, sodium EDTA, and heparin, plus one vial of traysolol or aprotinin (basic pancreatic trypsin inhibitor and heparin) per 300 mls of plasma, as will be known to those of skill in the art. The plasma is generally placed in an ice bath immediately. Next, the plasma may be transferred to a beaker in an ice bath with a stir bar and brought up to 0.001% PMSF (phenylmethylsulphonyl-fluoride) while stirring gently in the ice bath (PMSF can be dissolved to 10% in dry isopropyl alcohol and stored in dark bottle/vial in the freezer). The plasma is then overlayed with plasma with water (15% of plasma volume) and use low speed centrifugation at 10,000 rpms for 1 hr to remove chylomicrons. Vacuum is used to remove the chylomicron layer and the infranatant is placed in an ice bath. Discard any pellet present and repeat steps on the infranatant if necessary.

Next, the density is raised to 1.019 g/ml with potassium bromide or sodium bromide to remove both very low-density lipoproteins (VLDL) and intermediate-density lipoproteins (IDL) and the supernatant centrifuged at 40,000 rpms for 24 hrs at 4° Celsius, the lipoprotein layer is removed and the centrifugation step is repeated. Next, the density of the infranate is increased to 1.05 g/ml with KBr or NaBr to float LDL, which is then centrifuged at 40,000 rpms for 24 hrs at 4° C. The LDL layer is removed and the infranate discarded. Another density adjustment is conducted on the LDL fraction to a density 1.07 g/ml with salt and centrifuge at 40,000 rpms for 18 hrs at 4° C., followed by collection of the LDL fraction and place in ice bath.

Next, the LDL is dialyzed in 25 mM Na-phosphate buffer, pH 7.3. The LDL is usually dialyzed 1 to 80 vol/vol (e.g., 50 mls LDL in 4 liters of buffer), with a change of buffer about every 3 hrs at 4 C. Dialyze the fourth change in phosphate buffered saline solution for about 4° hours or overnight at 4° C. Finally, the purified LDL may be stores at 4 C. Generally, stores aliquots are overlaid with nitrogen gas or argon and the containers are well sealed. Upon opening an aliquot any remainder is most often discarded. Under normal storage conditions, LDL sample should be good for about two weeks. The integrity of, e.g., apo B is checked by running on a reduced-SDS PAGE gel. Use 4% or 5% polyacrylamide (make from 30% acrylamide and 0.8% bisacrylamide) for PAGE. Apply about 10 micrograms of apo B protein to the gel. Stain gels with coomassie brilliant blue. LDL-protein concentration can be determined using the SDS-Lowry method. There are other methods that will work in the presence of detergents, e.g., determinations with a monoclonal ELISA standard.

Alternatively, the LDL may be purified using chromatographic method for isolation of LDL. Briefly, fresh plasma is collected in a bag that includes, sodium azide, sodium citrate, sodium EDTA, and heparin, plus one vial of traysolol or aprotinin (basic pancreatic trypsin inhibitor and heparin) per 300 mls of plasma. The plasma is placed in an ice bath immediately. Next, the plasma is transferred to a beaker in an ice bath with a stir bar and up to 0.001% PMSF (phenylmethylsulphonyl-fluoride) added while stirring gently in the ice bath (PMSF can be dissolved to 10% in dry isopropyl alcohol and stored in dark bottle/vial in the freezer). Next, a semi-dry slurry of BioRad's Affi-gel Blue is prepared in phosphate-buffered saline in a beaker in a cold environment at 4-10° C. Plasma is added while stirring gentle either with a rod or by swirling the beaker, and allowed to stand for 20 minutes with occasional stirring. The slurry is poured into a filtered device, funnel or column and the filtrate collected. Albumin and plasminogen will generally be retrained on the gel and the filtrate will include plasma proteins including all lipoproteins except lipoprotein[a]. PMSF is added as before. The filtrate is run on a column of dextran-sulfate cellulose equilibrated with PBS containing 2 mM magnesium chloride; the column is washed with 2 bed volumes of PBS or until optical density at 280 nm is below 0.05. Only low density lipoproteins, LDL, IDL, and VLDL generally bind to this gel under these conditions. The LDL is then eluted with PBS containing 1.5 M NaCl and the LDL dialyze to lower ionic strength or pass thru a desalting column such as BioRad P10 gel. The lipoprotein fraction may also be applied to a DNA (nucleic acid) affinity gel column. The DNA will generally be a sequence that binds LDL such as the immediate-early 2 promoter of the human cytomegalovirus or an oligonucleotide such as ISRE or interferon-stimulated response element. LDL will bind to the column and may be removed with high ionic strength solutions. This step may also be performed using a slurry of the affinity gel as explained above.

LDL-DNA complex formation. In particular aspects of the present invention, lipoproteins are employed in order to transport DNA into cell in vitro and in vivo. In the present invention, optimal DNA/LDL binding has been established. In one embodiment, a 1:1 ratio of DNA:LDL protein molar ratio of 1:1 are incubated at 37° C. for 30 min in a buffered solution. An exemplary buffer may be 50 mM Tris-HCl at pH 7.4 containing 150 mM NaCl, and 10 mM MgCl₂. The concentrations of DNA and LDL protein may range from the picomolar range to the micromolar range. In one embodiment and equal amount of nucleic acid and lipoprotein are mixed, e.g., 0.39 pmole DNA are incubated with 0.39 pmole LDL-protein.

The incubation conditions may be altered to increase or decrease the efficiency of DNA/LDL binding. For example the incubation may occur at temperatures ranging from 4° C. to 50° C. The reaction mixture may be incubated at 4° C., 6° C., 8° C., 10° C., 12° C., 14° C., 16° C., 18° C., 20° C., 22° C., 24° C., 26° C., 28° C., 30° C., 32° C., 34° C., 36° C., 38° C., 40° C., 42° C., 44° C., 46° C., 48° C., 50° C. The time of incubation may be varied from as little as 10 minutes to as long as 5 hours. It is well within the skill of one in the art to incubate the mixture for varying degrees of time. Other embodiments contemplate varying the concentration of MgCl₂ in the media. Thus, the MgCl₂ concentration may vary from 1 mM to 100 mM. The reaction mixture may also include, e.g., 5 mM MgCl₂, 10 mM MgCl₂, 12 mM MgCl₂, 15 mM MgCl₂, 20 mM MgCl₂, 30 mM MgCl₂, 35 mM MgCl₂, 40 mM MgCl₂, 50 mM MgCl₂, 60 mM MgCl₂, 65 mM MgCl₂, 70 mM MgCl₂, 80 mM MgCl₂, 90 mM MgCl₂, 100 mM MgCl₂ or greater.

Gene delivery and expression in eukaryotic cells. The gene delivery system of the instant invention can be used to express any gene of interest in eukaryotic cells, and in particular antigens. As the skilled artisan will recognize, the selection of antigens for expression will be selected based on the need to trigger or suppress an immune response. For example, the antigen may be a cancer antigen, where the user will want to trigger a strong immune response based on providing intracellular expression of antigens that are pre-processed, processed within the cell or endocytosed and loaded onto an MHC molecule for presentation. For antigens that trigger an antibody response, the antigen may be expressed on cell surfaces, secreted or both for immune activation. Also, antigens that are expressed may provide for both presentation by MHC and triggering of B-cell responses. Furthermore, antigens may be provided that anergize or suppress an immune response. Antigens that suppress an immune response would be ideal to control antigens that cause or trigger, e.g., allergies, auto-immune disorders and the like.

The antigen gene, e.g., the isolated and purified cDNA sequence of the antigen (or portions thereof) is cloned into a plasmid containing the specific lipoprotein binding sequences (including, but not limited to SRE, E/C, FAS) and/or any eukaryotic regulatory sequence (for example, but not limited to HCMV, or tyrosine kinase promoter region) using DNA cloning techniques well known to the art. In some cases the antigen may already include such sequences, as such, none may have to be added to the antigen cDNA or may be added to enhance binding. The orientation, number and location of the lipoprotein binding sequences may vary within the nucleic acid vector, but should not interrupt the protein coding sequence of the gene of interest.

The gene delivery system of the instant invention can be used to transfect eukaryotic cells either in vivo or in vitro with any expression vector containing one or more of the aforementioned lipoprotein binding sequences. Expression vectors are designed using recombinant DNA cloning techniques known to the art and generally include five components linked in the following 5′ to 3′ orientation: i) an eukaryotic promoter sequence, 2) a sequence encoding a 5′ untranslated RNA (UTR) which may include a first intron sequence followed by a consensus Kozak sequence and an initiation ATG, 3) a protein coding sequence, 4) a 3′ UTR, and 5) a cognate transcription terminator sequence.

Lipoproteins are isolated from blood in a manner similar to the previously described procedures (see, Example 1) and bound to the nucleic acids of interest in a manner similar to the previously described DNA binding protocol (see, Example 2). Separation of protein-bound DNA from free DNA may be required prior to transfection and can be accomplished by adsorption to nitrocellulose membranes or other techniques well known to the art including, but not limited to size-exclusion or density ultracentrifugation.

Control Regions. In order for the gene delivery system of the present invention to effect expression of a transcript encoding a selected gene, the polynucleotides encoding these genes will be under the transcriptional control of a promoter. A “promoter” refers to a DNA sequence recognized by the synthetic machinery of the host cell, or introduced synthetic machinery, that is required to initiate the specific transcription of a gene. The phrase “under transcriptional control” means that the promoter is in the correct location in relation to the polynucleotide to control RNA polymerase initiation and expression of the polynucleotide.

The term promoter will be used here to refer to a group of transcriptional control modules that are clustered around the initiation site for RNA polymerase II. Much of the thinking about how promoters are organized derives from analyses of several viral promoters, including those for the HSV thymidine kinase (tk) and SV40 early transcription units. These studies, augmented by more recent work, have shown that promoters are composed of discrete functional modules, each consisting of approximately 7-20 bp of DNA, and containing one or more recognition sites for transcriptional activator or repressor proteins.

At least one module in each promoter functions to position the start site for RNA synthesis. The best known example of this is the TATA box, but in some promoters lacking a TATA box, such as the promoter for the mammalian terminal deoxynucleotidyl transferase gene and the promoter for the SV40 late genes, a discrete element overlying the start site itself helps to fix the place of initiation. Additional promoter elements regulate the frequency of transcriptional initiation. Typically, these are located in the region 30-110 bp upstream of the start site, although a number of, promoters have recently been shown to contain functional elements downstream of the start site as well. The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either cooperatively or independently to activate transcription.

The particular promoter that is employed to control the expression of a therapeutic gene is not believed to be critical, so long as it is capable of expressing the polynucleotide in the targeted cell. Thus, where a human cell is targeted, it is preferable to position the polynucleotide coding region adjacent to and under the control of a promoter that is capable of being expressed in a human cell. Generally speaking, such a promoter might include either a human or viral promoter. In one embodiment, the human cytomegalovirus (CMV) immediate early gene promoter, the SV40 early promoter and the Rous sarcoma virus long terminal repeat can be used to obtain high-level expression of the polynucleotide of interest. The use of other viral or mammalian cellular or bacterial phage promoters which are well-known in the art to achieve expression of polynucleotides is contemplated as well, provided that the levels of expression are sufficient to produce a growth inhibitory effect.

By employing a promoter with well-known properties, the level and pattern of expression of a polynucleotide following transfection can be optimized. For example, selection of a promoter which is active in specific cells, such as tyrosinase (melanoma), alpha-fetoprotein and albumin (liver tumors), CC10 (lung tumor) and prostate-specific antigen (prostate tumor) will permit tissue-specific expression of the therapeutic gene.

Enhancers were originally detected as genetic elements that increased transcription from a promoter located at a distant position on the same molecule of DNA. The ability of enhancers to act over a large distance had little precedent in classic studies of prokaryotic transcriptional regulation. Regions of DNA with enhancer activity are organized much like promoters, that is, enhancers may include many individual elements, each of which binds to one or more transcriptional proteins.

The basic distinction between enhancers and promoters is operational. An enhancer region as a whole must be able to stimulate transcription at a distance; this need not be true of a promoter region or its component elements. On the other hand, a promoter must have one or more elements that direct initiation of RNA synthesis at a particular site and in a particular orientation, whereas enhancers lack these specificities. Promoters and enhancers are often overlapping and contiguous, often seeming to have a very similar modular organization. Additionally, any promoter/enhancer combination (see e.g., Eukaryotic Promoter Data Base EPDB) may be used to drive expression of a particular construct. Use of a T3, T7 or SP6 cytoplasmic expression system is another possible embodiment. Eukaryotic cells can support cytoplasmic transcription from certain bacteriophage promoters if the appropriate bacteriophage polymerase is provided, either as part of the delivery complex or as an additional genetic expression vector. According to the present invention, a number of different promoters may be used. These promoters may be the same or different, but the selection of particular promoters for particular uses may be advantageous.

IRES. In certain embodiments of the invention, the antigen expressing genes may be placed 5′ from internal ribosome binding site (IRES) elements. These elements are used to create multigene, or polycistronic, messages. IRES elements are able to bypass the ribosome scanning model of 5′ methylated Cap dependent translation and begin translation at internal sites (Pelletier and Sonenberg, 1988). IRES elements from two members of the picornavirus family (polio and encephalomyocarditis) have been described (Pelletier and Sonenberg, 1988), as well an IRES from a mammalian message (Macejak and Sarnow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message.

Any heterologous open reading frame can be linked to IRES elements. This includes genes for secreted proteins, multi-subunit proteins, encoded by independent genes, intracellular or membrane-bound proteins and selectable markers. In this way, expression of several proteins can occur simultaneously in cell with a single construct and, e.g., a single selectable or detectable marker. In addition, it may be desirable to include polyadenylation signals in the vectors. These signals serve to terminate transcription and to stabilize mRNA transcripts produced from the vectors. One such polyadenylation signal is an SV40 polyadenylation signal.

Genes. The present invention contemplates the use of a variety of different genes inserted into, e.g., the SV40 vector. For example, genes encoding enzymes, hormones, cytokines, oncogenes, receptors, tumor suppressors, transcription factors, drug selectable markers, toxins and various antigens are contemplated as suitable.

In another example, the expression vector may include a nucleotide sequence encoding for functional apolipoprotein A-I (Apo A-I) for the prevention or treatment of artherosclerosis. Atherosclerosis is a disease that is characterized by the development of atherosclerotic lesions which contain cholesterol esters and other lipids that are derived from the blood circulation. The plasma concentration of HDL is inversely correlated with the risk for development of atherosclerosis. HDL present in the blood circulation take up free cholesterol from extrahepatic cells which through the action of LCAT (lecithin-cholesterol acyltransferase) is converted to cholesterol esters and stored in the core of the HDL particles. The HDL cholesterol esters are transported either directly or indirectly via transfer to triglyceride rich lipoproteins (i.e., VLDL, IDL, LDL) to the liver by a process called “reverse cholesterol transport”. Reverse cholesterol transport is of great importance for maintaining cholesterol homeostasis since the liver is the major organ for cholesterol excretion from the body via bile acids. Apo A-I is the major protein constituent of HDL and a cofactor LCAT. Therefore, increasing the plasma concentration of apo A-I containing HDL can increase the reverse cholesterol transport and reduce the risk for atherosclerosis.

Antigenic targets that may be delivered using the DNA LDL vaccines of the present invention include genes encoding antigens such as viral antigens, bacterial antigens, fungal antigens or parasitic antigens. Viruses include picornavirus, coronavirus, togavirus, flavirvirus, rhabdovirus, paramyxovirus, orthomyxovirus, bunyavirus, arenavirus, reovirus, retrovirus, papilomavirus, parvovirus, herpesvirus, poxvirus, hepadnavirus, and spongiform virus. Other viral targets include influenza, herpes simplex virus 1 and 2, measles, dengue, smallpox, polio or HIV. Pathogens include trypanosomes, tapeworms, roundworms, helminthes, malaria. Tumor markers, such as fetal antigen or prostate specific antigen, may be targeted in this manner. Other examples include: HIV env proteins and hepatitis B surface antigen. Administration of a vector according to the present invention for vaccination purposes would require that the vector-associated antigens be sufficiently non-immunogenic to enable long term expression of the transgene, for which a strong immune response would be desired. In some cases, vaccination of an individual may only be required infrequently, such as yearly or biennially, and provide long term immunologic protection against the infectious agent. Specific examples of organisms, allergens and nucleic and amino sequences for use in vectors and ultimately as antigens with the present invention may be found in U.S. Pat. No. 6,541,011, relevant portions incorporated herein by reference, in particular, the tables that match organisms and specific sequences that may be used with the present invention.

Plasmid DNA vaccines for malaria that include one or more of the following recombinant genes or fusion protein chimeras can be delivered using the LDL vector: CSP-1, STARP, SALSA, SSP-2, LSA-1, EXP-1, LSA-3, RAP-1, RAP-2, SERA-1, MSP-1, MSP-2, MSP-3, MSP-4, MSP-5, AMA-1, EBA-175, Pf35, Pf55, RESA, EMP-1, GLURP, Pfs16, Pfs25, Pfs28, Pfs45, Pfs48, Pfs230, Pfg27, and Pfs28.

Bacterial Pathogens; antibiotic resistance, e.g., haemophilus influenza; Plasmodium falciparum; neisseria meningitidis; streptococcus pneumoniae; neisseria gonorrhoeae; salmonella serotype typhi; shigella; vibrio cholerae; Dengue Fever; Encephalitides; Japanese Encephalitis; lyme disease; Yersinia pestis; west nile virus; yellow fever; tularemia; hepatitis (viral; bacterial); RSV (respiratory syncytial virus); HPIV 1 and HPIV 3; adenovirus; small pox; allergies and cancers.

It may be advantageous to combine portions of genomic DNA with cDNA or synthetic sequences to generate specific constructs. For example, where an intron is desired in the ultimate construct, a genomic clone will need to be used. The cDNA or a synthesized polynucleotide may provide more convenient restriction sites for the remaining portion of the construct and, therefore, would be used for the rest of the sequence.

Self-initiating and self-sustaining gene expression systems. The invention gene delivery system can also be used to delivery self-initiating and self-sustaining gene expression systems. Self-initiating and self-sustaining gene expression systems may be constructed by binding a RNA polymerase to a DNA construct in vitro prior to the introduction of the polynucleotide into the cell as described by Wagner, et al. (U.S. Pat. No. 5,591,601, relevant portions incorporated herein by reference). The RNA polymerase is bound to a DNA construct containing a cognate promoter of the RNA polymerase linked operably to a DNA sequence encoding for the RNA polymerase.

The expression of functional RNA polymerase in turn enables the expression of any gene of interest that contains a cognate promoter sequence recognized by the same RNA polymerase in eukaryotic host cells. DNA sequences encoding for both RNA polymerase and gene product of interest (i.e., protein of interest) may be contained within the same gene expression system. The gene expression system may be pre-bound to purified plasma lipoprotein fractions prior to transfection into eukaryotic cells.

Delivery of DNA to Cells in vivo. The invention gene delivery system can also be used to deliver DNA to cells in vivo. An expression vector containing the polynucleotide sequences of the gene of interest (e.g., reporter gene or a healthy copy of a defective gene) is prebound to LDL according to the protocols described herein. The DNA-LDL complex is then introduce into an organism for example, a rat, mouse or human by, for example, intravenous injection. At varying times post-injection, LDL is isolated from the blood and probed for DNA sequences of the type that were pre-bound to the LDL using standard molecular biological techniques such as, but not limited to, Southern blot hybridization or PCR™.

The LDL may also be immunoprecipitated with anti-LDL antibodies and then probed for specific DNA sequences bound to it. In order to determine cellular internalization and/or integration of the reporter gene sequences into the genomic DNA of cells of different tissues, total genomic DNA can be isolated from various tissues (according to standard molecular biology techniques) and probed for the presence of the reporter gene sequences using specific polynucleotide probes in PCR™ or Southern blot hybridization techniques. In addition, total cellular RNA can be isolated from various different tissues using standard molecular biology techniques and probed for the presence of specific mRNA encoded for by the reporter gene polynucleotide sequences using specific antisense polynucleotide probes in Northern blot hybridization techniques or ribonuclease (RNase) protection assays.

Expression of a functional protein encoded for by the gene of interest in different tissues can be analyzed using techniques well known to the art, such as, Western blot hybridization of cellular protein extracts with antibodies that bind specifically to the reporter gene product (i.e., protein of interest) or direct detection of intracellular fluorescence (e.g., when reporter genes are used that encode for blue or green fluorescent proteins.

Once the DNA-LDL complex has been delivered into the cell, the nucleic acid encoding the gene of interest may be positioned and expressed at different sites. In certain embodiments, the nucleic acid encoding the gene may be integrated stably into the genome of the cell. This integration may be in the cognate location and orientation via homologous recombination (gene replacement) or it may be integrated in a random, non-specific location (gene augmentation). In yet further embodiments, the nucleic acid may be stably maintained in the cell as a separate, episomal segment of DNA. Such nucleic acid segments or “episomes” encode sequences sufficient to permit maintenance and replication independent of or in synchronization with the host cell cycle. How the DNA-LDL complex is delivered to a cell and where in the cell the nucleic acid remains is dependent on the type of DNA molecule bound to the LDL.

Selected organs including the liver, skin, and muscle tissue of rats and mice have been bombarded in vivo (Yang et al., 1990; Zelenin et al., 1991). Bombarded in vivo may require surgical exposure of the tissue or cells, to eliminate any intervening tissue between the gun and the target organ, i.e., ex vivo treatment. Again, DNA encoding a particular gene may be delivered via this method and still be incorporated by the present invention. In a further embodiment of the invention, the DNA-LDL complex may be entrapped in a liposome. Liposomes are vesicular structures characterized by a phospholipid bilayer membrane and an inner aqueous medium. Multilamellar liposomes have multiple lipid layers separated by aqueous medium, which form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). Also contemplated are lipofectamine-DNA complexes.

Liposome-mediated nucleic acid delivery and expression of foreign DNA in vitro has been very successful. Wong, et al., (1980) demonstrated the feasibility of liposome-mediated delivery and expression of foreign DNA in cultured chick embryo, HeLa and hepatoma cells. Nicolau, et al., (1987) accomplished successful liposome-mediated gene transfer in rats after intravenous injection. In certain embodiments of the invention, the liposome may be complexed with a hemagglutinating virus (HVJ). This has been shown to facilitate fusion with the cell membrane and promote cell entry of liposome-encapsulated DNA (Kaneda et al., 1989). In other embodiments, the liposome may be complexed or employed in conjunction with nuclear non histone chromosomal proteins (HMG-1) (Kato, et al., 1991). In yet further embodiments, the liposome may be complexed or employed in conjunction with both HVJ and HMG-1. In that such expression constructs have been successfully employed in transfer and expression of nucleic acid in vitro and in vivo, then they are applicable for the present invention. Where a bacterial promoter is employed in the DNA construct, it also will be desirable to include within the liposome an appropriate bacterial polymerase.

In certain embodiments, gene transfer may more easily be performed under ex vivo conditions. Ex vivo gene therapy refers to the isolation of cells from an animal, the delivery of a nucleic acid into the cells in vitro, and then the return of the modified cells back into an animal. This may involve the surgical removal of tissue/organs from an animal or the primary culture of cells and tissues. Anderson, et al., U.S. Pat. No. 5,399,346, relevant portions incorporated herein, disclose ex vivo therapeutic methods.

Pharmaceutical preparations. The gene delivery system of the instant invention can be administered in vivo in various ways including, but not limited to, intravenous, pharyngeal, epidermal, intramuscular, intraperitoneal (IP), nasal, and/or rectal. The gene delivery system of may also be used for in vitro transfection of eukaryotic cell types that possess specific lipoprotein receptors on their cytoplasmic membranes, but are not limited to these cell types.

Pharmaceutical products that may spring from the current invention may include a naked polynucleotide with a single or multiple copies of the specific nucleotide sequences that bind to specific DNA-binding sites of the apolipoproteins present on plasma lipoproteins as described in the current invention. The polynucleotide may encode a biologically active peptide, antisense RNA, or ribozyme and will be provided in a physiologically acceptable administrable form. Another pharmaceutical product that may spring from the current invention may include a highly purified plasma lipoprotein fraction, isolated according to the methodology, described herein from either the patients blood or other source, and a polynucleotide containing single or multiple copies of the specific nucleotide sequences that bind to specific DNA-binding sites of the apolipoproteins present on plasma lipoproteins, prebound to the.purified lipoprotein fraction in a physiologically acceptable, administrable form.

Yet another pharmaceutical product may include a highly purified plasma lipoprotein fraction which contains recombinant apolipoprotein fragments containing single or multiple copies of specific DNA-binding motifs, prebound to a polynucleotide containing single or multiple copies of the specific nucleotide sequences, in a physiologically acceptable administrable form. Yet another pharmaceutical product may include a highly purified plasma lipoprotein fraction which contains recombinant apolipoprotein fragments containing single or multiple copies of specific DNA-binding motifs, prebound to a polynucleotide containing single or multiple copies of the specific nucleotide sequences, in a physiologically acceptable administrable form.

The dosage to be administered depends to a great extent on the body weight and physical condition of the subject being treated as well as the route of administration and frequency of treatment. A pharmaceutical composition that includes the naked polynucleotide prebound to a highly purified lipoprotein fraction may be administered in amounts ranging from 1 μg to 1 mg polynucleotide and 1 μg to 100 mg protein.

Administration of the therapeutic virus particle to a patient will follow general protocols for the administration of chemotherapeutics, taking into account the toxicity, if any, of the vector. It is anticipated that the treatment cycles would be repeated as necessary. It also is contemplated that various standard therapies, as well as surgical intervention, may be applied in combination with the described gene therapy.

Where clinical application of a gene therapy is contemplated, it will be necessary to prepare the complex as a pharmaceutical composition appropriate for the intended application. Generally this will entail preparing a pharmaceutical composition that is essentially free of pyrogens, as well as any other impurities that could be harmful to humans or animals. One also will generally desire to employ appropriate salts and buffers to render the complex stable and allow for complex uptake by target cells.

Aqueous compositions of the present invention may include an effective amount of the compound, dissolved or dispersed in a pharmaceutically acceptable carrier or aqueous medium. Such compositions can also be referred to as inocula. The phrases “pharmaceutically or pharmacologically acceptable” refer to molecular entities and compositions that do not produce an adverse, allergic or other untoward reaction when administered to an animal, or a human, as appropriate. As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients also can be incorporated into the compositions. The compositions of the present invention may include classic pharmaceutical preparations. Dispersions also can be prepared in glycerol, liquid polyethylene glycols, and mixtures thereof and in oils. Under ordinary conditions of storage and use, these preparations contain a preservative to prevent the growth of microorganisms.

Disease States. Depending on the particular disease to be treated, administration of therapeutic compositions according to the present invention will be via any common route so long as the target tissue is available via that route in order to maximize the delivery of antigen to a site for maximum (or in some cases minimum) immune response. Administration will generally be by orthotopic, intradermal, subcutaneous, intramuscular, intraperitoneal or intravenous injection. Other areas for delivery include: oral, nasal, buccal, rectal, vaginal or topical. Topical administration would be particularly advantageous for treatment of skin cancers. Such compositions would normally be administered as pharmaceutically acceptable compositions that include physiologically acceptable carriers, buffers or other excipients.

In certain embodiments, ex vivo therapies also are contemplated. Ex vivo therapies involve the removal, from a patient, of target cells. The cells are treated outside the patient's body and then returned. One example of ex vivo therapy would involve a variation of autologous bone marrow transplant. Many times, ABMT fails because some cancer cells are present in the withdrawn bone marrow, and return of the bone marrow to the treated patient results in repopulation of the patient with cancer cells. In one embodiment, however, the withdrawn bone marrow cells could be treated while outside the patient with an LDL-DNA particle that targets and kills the cancer cell. Once the bone marrow cells are “purged,” they can be reintroduced into the patient.

The treatments may include various “unit doses.” Unit dose is defined as containing a predetermined-quantity of the therapeutic composition calculated to produce the desired responses in association with its administration, i.e., the appropriate route and treatment regimen. The quantity to be administered, and the particular route and formulation, are within the skill of those in the clinical arts. The subject to be treated may also be evaluated, in particular, the state of the subject's immune system and the protection desired. A unit dose need not be administered as a single injection but may include continuous infusion over a set period of time. Unit dose of the present invention may conveniently may be described in terms of 0.01 mg DNA/kg body weight to 0.4 mg DNA/kg body weight, with ranges in between these being contemplated such that 0.05, 0.10, 0.15, 0.20, 0.25, 0.5 mg/DNA/kg body weight are administered. Likewise the amount of LDL delivered can vary from about 0.2 to about 8.0 mg/kg body weight. Thus, in particular embodiments, 0.4 mg, 0.5 mg, 0.8 mg, 1.0 mg, 1.5 mg, 2.0 mg, 2.5 mg, 3.0 mg, 4.0 mg, 5.0 mg, 5.5 mg, 6.0 mg, 6.5 mg, 7.0 mg and 7.5 mg of LDL may be delivered to an individual in vivo. The dosage of DNA:LDL to be administered depends to a great extent on the weight and physical condition of the subject being treated as well as the route of administration and the frequency of treatment. A pharmaceutical composition that includes a naked polynucleotide prebound to a highly purified lipoprotein fraction may be administered in amounts ranging from 1 μg to 1 mg polynucleotide to 1 μg to 100 mg protein. Thus, particular compositions may include between about 1 μg, 5 μg, 10 μg, 20 μg, 30 μg, 40 μg, 50 μg, 60 μg, 70 μg, 80 μg, 100 μg, 150 μg, 200 μg, 250 μg, 500 μg, 600 μg, 700 μg, 800 μg, 900 μg or 1000 μg polynucleotide that is bound independently to 1 μg, 5 μg, 10 μg, 20 μg, 3.0 μg, 40 μg, 50 μg, 60 μg, 70 μg, 80 μg, 100 μg, 150 μg, 200 μg, 250 μg, 500 μg, 600 μg, 700 μg, 800 μg, 900 μg, 1 mg, 1.5 mg, 5 mg, 10 mg, 20 mg, 30 mg, 40 mg, 50 mg, 60 mg, 70 mg, 80 mg, 90 mg or 100 mg lipoprotein. Any amount of polynucleotide may be bound to any other amount of lipoprotein to achieve the pharmaceutical concentrations of the present invention.

Cancer Antigens. One of the embodiments of the present invention involves the use of the LDL vectors to deliver a nucleic acid that encodes one or more cancer antigens to cells. Target cells include lung, brain, prostate, kidney, liver, ovary, breast, skin, stomach, esophagus, head & neck, testicles, colon, cervix, lymphatic system and blood. Of particular interest are antigens for non-small cell lung carcinomas including squamous cell carcinomas, adenocarcinomas and large cell undifferentiated carcinomas.

The terms “contacted” and “exposed,” when applied to a cell, are used herein to describe the process by which a therapeutic construct and a chemotherapeutic or radiotherapeutic agent are delivered to a target cell or are placed in direct juxtaposition with the target cell. To achieve cell killing or stasis, both agents are delivered to a cell in a combined amount effective to kill the cell or prevent it from dividing.

The therapeutic compositions of the present invention are advantageously administered in the form of injectable compositions either as liquid solutions or suspensions; solid forms suitable for solution in, or suspension in, liquid prior to injection may also be prepared. These preparations also may be emulsified. A typical composition for such purpose includes a pharmaceutically acceptable carrier. For instance, the composition may contain 10 mg, 25 mg, 50 mg or up to about 100 mg of human serum albumin per milliliter of phosphate buffered saline.

Other pharmaceutically acceptable carriers include aqueous solutions, non-toxic excipients, including salts, preservatives, buffers and the like. Examples of non-aqueous solvents are propylene glycol, polyethylene glycol, vegetable oil and injectable organic esters such as ethyloleate. Aqueous carriers include water, alcoholic/aqueous solutions, saline solutions, parenteral vehicles such as sodium chloride, Ringer's dextrose, etc. Intravenous vehicles include fluid and nutrient replenishers. Preservatives include antimicrobial agents, anti-oxidants, chelating agents and inert gases. The pH and exact concentration of the various components the pharmaceutical composition are adjusted according to well known parameters.

Additional formulations are suitable for oral administration. Oral formulations include such, typical excipients as, for example, pharmaceutical grades of mannitol, lactose, starch, magnesium stearate, sodium saccharine, cellulose, magnesium carbonate and the like. The compositions take the form of solutions, suspensions, tablets, pills, capsules, sustained release formulations or powders. When the route is topical, the form may be a cream, ointment, salve or spray.

Example 1—Materials and Methods—Isolation of Plasma Lipoproteins. Restriction endonucleases were purchased from Life Technologies, and Protease inhibitors (i.e., leupeptin, PMSF, and Trasylol) were purchased from Sigma Chemical Company. Plasma lipoproteins were isolated using standard sequential flotation ultracentrifugation methods as described (Schumaker and Puppione, 1986). Throughout the entire procedure samples were kept on ice or at 4° C. unless otherwise stated.

Subjects were fasted for at least 4 h prior to the start of the procedures: Blood was drawn into sterile, vacuumed glass tubes containing anticoagulants, e.g., 0.1% (ethylenedinitrolo)-tetracetic acid (EDTA) or heparin. Plasma was obtained by centrifugation (10 minutes at 3000×g) and immediately adjusted to 0.005% phenylmethansulfonyl fluoride (PMSF), 10 KIU Trasylol/ml, and 1 μg leupeptin/ml. VLDL, LDL, and HDL fractions were isolated by sequential flotation ultracentrifugation for 18 h at 40,000 rpm in a Beckmann centrifuge Model LS-80M after plasma samples were adjusted with potassium bromide (ICBr) to solution densities of 1.006, 1.019, and 1.215 g/ml respectively. Immediately following ultracentrifugation, individual lipoprotein fractions were collected and dialyzed extensively against phosphate buffered saline (pH 7.4) containing 0.001% sodium azide. Protein concentrations were determined using standard BCA protein assays (Pierce Chemical Company).

DNA-Binding Protocol. Lipoproteins and DNA were mixed together and incubated for 30 min at room temperature in 50 mmole/liter Tris (pH 7.4), 100-154 mmoles/liter sodium chloride (NaCl), 15 mmoles/liter magnesium chloride (MgCl₂) six times. Sample loading buffer (30% glycerol, 0.25% Xylene cyanole FF, 0.25% bromophenol blue) was added to the samples in a 1:5 V/V ratio. Samples were underloaded into 30 μl wells at the cathode edge of an 0.8% agarose gel containing 1 μg ethidium bromide/ml in Tris-Acetate buffer (pH 7.85) and electrophoresis was accomplished using 100 Volt constant until the negatively charged tracking dye had migrated at least 50% of distance from the loading well to the end of the gel closest to the anode.

Agarose Electrophoretogram of Human Lipoproteins. Agarose electrophoresis of human lipoproteins has been performed to illustrating the differential migration patterns of lipoprotein fractions VLDL, LDL, and HDL isolated from human plasma resolved using non-denaturing conditions. Plasma lipoproteins were isolated from human blood according to the protocol described above, in which 6×Sample loading buffer (30% glycerol, 0.25% Xylene cyanole FF, 0.25% bromophenol blue) was added to the samples in a 1:5 V/V ratio. Samples were underloaded into 30 μl wells at the cathode edge of an 0.8% agarose gel in Tris-Acetate buffer (pH 7.85) and electrophoresis was accomplished using 100 Volt constant until the negatively charged tracking dye had migrated at least 50% of the distance from the loading well to the anodic edge of the gel.

Following electrophoresis, the agarose gel was stained for protein in a solution containing 50% V/V ethanol, 10% V/V acetic acid, and 0.25% Coomasie Brilliant Blue R-250 (CBB R-250, Bio-Rad Labs). Lane 1 contained human VLDL (10 μg protein), Lane 2 contained human LDL (35 μg protein), and Lane 3 contained human HDL (35 μg protein). Results illustrated the differential migration of lipoprotein fractions, VLDL, LDL, and HDL, isolated from human plasma resolved using non-denaturing conditions by agarose gel electrophoresis. Lipoproteins were visualized using a protein binding dye, Coomassie Brilliant Blue (CBB). The absence of other bands in each lane indicated the high degree of purity for each lipoprotein.

Labeling of Deoxyoligonucleotides. Complementary single stranded oligonucleotides were mixed (10 μg each) and incubated at 85° C. for 5 min in 10 mM Tris HCl (pH 7.4). Immediately following incubation, the samples were cooled down slowly to room temperature to obtain double stranded oligonucleotides. The double stranded oligonucleotides were then digested with BamHI and EcoRI for 1 h at 37° C. in 50 mM Tris HCl (pH 8.0), 100 mM NAG1, and 10 mM MgCl₂. Digested double stranded oligonucleotides were purified using a Qiaquick nucleotide removal kit from Qiagen Inc. according to manufacturer's protocol. The 5′ protruding ends of the purified oligonucleotides were then labeled with ³²P-α dATP using a Prime-It II labeling kit containing Exo (−) Klenow enzyme from Stratagene Inc. according to the manufacturer's protocol. The specific activity of all oligonucleotides was determined by scintillation counting.

The DNA-binding studies were performed as described above except that the agarose gel was not stained with ethidium bromide. Instead, following electrophoresis, the agarose gel was dried under vacuum and exposed to X-ray film for 4 h at room temperature prior to protein staining in a solution containing 50% V/V ethanol, 10% V/V acetic acid, and 0.25% Coomassie Brilliant Blue R-250 (Bio-Rad Labs). Oligonucleotides and human LDL were present at 400,000 cpm and 40 μg protein per lane respectively.

Sonication of Plasma Lipoproteins. Solutions of plasma lipoproteins in phosphate-buffered saline containing 10 mM MgCl₂ were kept on ice and sonicated for various time periods ranging from 0 to 6 minutes in a Sonifier Model 350 sonicator (Branson Sonic Power Co.) at the following settings: duty cycle; 30%, pulsed, output control; level 2. Immediately following sonication, genomic DNA was added to the sonicated solutions, and the DNA-binding assay (see above) was started.

RT-PCR™ of Lipoprotein-bound RNA. Human liver RNA, complexed to human LDL or to human VLDL as described above, was subjected to agarose gel electrophoresis and extracted from the gel by solubilizing the gel for 20 min at 50° C. in 3 times the gel volume of QX-1 buffer (Qiagen) and by twice adding an equivalent volume of phenol/chloroform (pH 4.0). RNA was precipitated by adding an equivalent volume of 100% isopropanol and freezing the mixture overnight at −80° C. RNA pellets were dissolved in 50 μl of DEPC-treated water. For each reaction, the dissolved RNA (3 μl) was transcribed in reverse into single-stranded DNA by adding 100 mM KCl, 10 mM Tris-HCl (pH 8.3), 5 mM MgCl₂, 2.5 μM primer (oligo d(T) or random hexamers), 1 U/μl RNase inhibitor, 1 mM each of dATP, dCTP, dTTP, and dGTP, and 2.5 U/μl of MMLV reverse transcriptase in a total reaction volume of 20 μl. The single-stranded DNA samples were then amplified in 100 mM KCl, 10 mM Tris-HCl (pH 8.3), 2 mM MgCl₂, 0.15 μM each of the forward and reverse ISRE primers (see Table 2), 1 mM each of dATP, dCTP, dTTP, and dGTP, and 2.5 U/100 μl of AmpliTaq DNA polymerase in a total reaction volume of 100 μl. DNA amplification was carried out in a thermocycler in 30 consecutive cycles of denaturing at 95° C. for 60 sec, reannealing at 55° C. for 60 sec, primer extension at 72° C. for 120 sec, and a final extension at 72° C. for 7 min. For each PCR reaction, 10 μl of the reaction mixture was analyzed by electrophoresis on a 1% agarose gel in TBE buffer (45 mM Tris-borate and 1 mM EDTA, pH 8.0) while maintaining a 100-V constant for 1 h. The PCR products were visualized by staining the gel with ethidium bromide.

DNA Sequencing. DNA fragments obtained from the RT-PCR reactions were separated by electrophoresis on a 1% agarose gel and extracted from the gel by using a Qiagen gel extraction kit according to the manufacturer's protocol. DNA samples were analyzed on an Applied Biosystems Inc. Model 373 automated DNA sequence apparatus after dye-terminator thermo cycle sequencing.

Cell Culture and Transfection Assays. Human skin fibroblasts were cultured in complete growth medium consisting of Dulbecco's modified Eagle's medium that was supplemented with 10% fetal bovine serum, 100 μg/ml each of streptomycin and penicillin at 37° C. in an atmosphere of 5% CO.sub.2 in a humidified incubator. Twenty-four hours before cell transfection, during exponential growth, the cultured cells were harvested by trypsinization, replated at a cell density of 1×10⁶ cells in 35-mm culture dishes containing a glass coverslip, and cultured in complete growth medium. All transfection studies were performed in triplicate as described.

LDL Assay. The pEGFP-N1 plasmid and LDL were mixed together at a ratio of 1:10 (wt/wt) in 100 μl of serum-free medium containing 10 mM MgCl₂ and incubated for 15 min at 37° C. When the cells were 40 to 60% confluent, they were transfected for 16 h at 37° C. with a mixture of 5 μg of DNA and 50 μg of LDL per 35-mm culture dish, each dish having been diluted in 1 ml of serum-free medium. Upon transfection, the LDLs were removed by gentle washing and maintained in 2 ml of growth medium per 35-mm culture dish for 24 h at 37° C. At 24 h post-transfection, the cells were washed with PBS and fixed in 2 ml of PBS containing 4% paraformaldehyde per 35-mm culture dish for 30 min. The coverslips were removed from the culture dishes, washed with PBS, placed in an inverted orientation on glass slides, and examined by fluorescent microscopy to detect GFP.

In vivo Reporter Gene Expression. Two-month-old female Sprague-Dawley rats were anesthetized with a combination anesthetic (42.8 mg/ml ketamine, 8.6 mg/ml xylazine, and 1.4 mg/ml acepromazine), and a prebound complex of purified rat LDL and linearized pEGFP-N1 plasmid DNA was injected intravenously (into the femoral vein), subcutaneously, intraperitoneally, and into the pharyngeal, nasal, and rectal mucosae (100 μg of LDL protein and 5 μg of DNA in 100 μl of PBS containing 10 mM MgCl₂ per site). Control animals were injected with linearized pEGFP-N1 plasmid DNA in which the HCMV IE promoter sequence was interrupted only by digestion with restriction enzymes. Next, 5 μg of DNA in 100 μl of PBS containing 10 mM MgCl₂ were added per site. After 2, 5 or 7 days, all the treated and control rats were sacrificed, their blood was collected by means of cardiac puncture, and the tissues were excised and immobilized in OCT by means of snap freezing over liquid nitrogen or by immediate freezing in liquid nitrogen. The immobilized tissue samples were sectioned on a cryomicrotome, and the sections (5-8 μm thick) were fixed for 30 min in 4% paraformaldehyde and analyzed for expression of EGFP (green fluorescent protein) by fluorescent microscopy.

Fluorescent Microscopy. Microscopy was performed by using an Olympus Model BH-2 fluorescent microscope (Olympus, USA) equipped with a digital camera (Hamamatsu, Model C5810) and a color printer (Image Master, Toshiba). The filter set used was a standard fluorescein isothiocyanate (FITC) set (Chroma Technology, Brattleboro, Vt., USA). The maximum excitation and emission wavelengths for this filter set were 485 nm (range 460-510 nm) and 540 nm (range 515-565 nm), respectively. Transfection efficiency was determined by calculating the average percentage of transduced cells of five different fields per 35-mm culture dish.

Detection of GFP. Excised rat tissues were homogenized in 150 μl of PBS in a dounce homogenizer placed on ice. The homogenized tissues were centrifuged for 3 min at 13,000×g, and 50-μl aliquots were withdrawn and used in an ELISA assay to detect GFP. First, serial dilutions (range 1:10 to 1:1,000) of all samples were made in PBS. ELISA plates (96 wells) were coated with the samples (three wells/sample) by incubating the plates at room temperature for 3 h. The plated samples were then washed three times with 200 μl of 1×PBS containing 0.1% Tween 20 (PBST) and blocked with 200 μl of PBST containing 1% bovine serum albumin (BSA) for 2 h at room temperature while shaking gently. The washing procedure was repeated with 200 μl of PBST containing 0.1% BSA, and the plated samples were incubated with a 1:2,000 dilution of a recombinant GFP polyclonal antibody (IgG fraction, Clontech Inc., Palo Alto, Calif.) in PBST containing 0.1% BSA (50 μl of diluted mixture per well) for 18 h at 4° C. while shaking gently. The plated samples were washed and incubated with a 1:5000 dilution of HRP-conjugated goat anti-rabbit antibody (IgG fraction, Cappel, Durham, N.C.) in PBST containing 0.1% BSA for 1 h at room temperature while shaking gently. The washing procedure was repeated and was followed by a final wash with 1×PBS. GFP was detected after a 30-minute incubation at room temperature in PBS containing σ-phenylenediamine as a chromogenic substrate.

Example 2. Binding of Genomic DNA to Human LDL. The binding of human genomic DNA (hg DNA) to human LDL has also been demonstrated. Each lane of the agarose gel contained hg DNA cut with AluI or HindIII. In addition, human VLDL and mouse LDL were run alongside the hg DNA. Plasma lipoproteins were isolated from human or mouse blood according to the protocol described above. DNA-binding studies were performed using human genomic DNA digested with either AluI or HindIII. Following electrophoresis, the gel was stained for DNA with ethidium bromide prior to protein staining in a solution containing 50% V/V ethanol, 10% V/V acetic acid, and 0.25% Coomasie Brilliant Blue R-250 (CBB R-250, Bio-Rad Labs). Each lane contained 5 μg human genomic DNA (hg DNA) cut with AluI or HindIII. In addition, human VLDL (10 μg protein per lane) human LDL (35 μg protein per lane) and mouse LDL (10 μg protein per lane) were also analyzed.

Bands in this study showed specific binding of digested human DNA fragments and human LDL by gel-shift electrophoresis. DNA fragment obtained by AluI or HindIII digestion of human genomic DNA are shown to migrate toward the anode with much slower mobility when preincubated with human LDL but not when incubated with human VLDL, human HDL, or mouse LDL. The complexed DNA/lipoprotein band is first visualized using DNA-binding ethidium bromide and photographed using transmitted ultra-violet light for activation of the fluorescent dye. Lipoproteins were next visualized with CBB and photographed using transmitted visible light. The results shown in this figure indicate that aliquot of AluI- and HindIII-digested human genomic DNA fragments comigrate with human LDL and are therefore bound to human LDL.

Example 3. Low-density Lipoprotein Interacts With Human Cytomegalovirus Genomic DNA. DNA binding studies with purified plasma lipoprotein fractions and human genomic DNA, as well as several different plasmids, demonstrated that purified LDL binds to human genomic DNA digested with different restriction enzymes (AluI and HindIII).

Purified LDL also bound to several different plasmids but its binding affinity for plasmid DNA containing the HCMV IE promoter region was significantly higher. It was shown that the binding of both LDL and VLDL to the HCMV IE promotor region and SRE, MSRE, ISRE, MISRE, E/C, FAS, and MFAS oligonucleotides. The E/C oligonucleotide was used in these DNA binding studies because this oligonucleotide contains both a binding site for members of the C/EBP transcription factor family, which are involved in the regulation of differentiation-dependent adipocyte gene expression, as well as an overlapping E-box motif which is generally recognized by the eukaryotic basic helix-loop-helix (b-HLH) transcriptional regulators. LDL clearly has a greater affinity for all of the oligonucleotides tested than do VLDL. This is most likely due to interference with protein-DNA interaction caused by either the presence of other apolipoproteins on the surface of VLDL or an increased net charge as a result of the increased lipid content of VLDL.

The sequence specificity is illustrated by the fact that both LDL and VLDL show a decreased binding affinity for the mutated versions of the ISRE and FAS oligos (MISRE and MFAS respectively). In contrast, LDL showed an increased binding affinity for the mutated version of the SRE oligo (MSRE). It is possible that this mutated SRE sequence may be a better ligand for the putative DNA binding region of apo B present on LDL. The binding of both VLDL and LDL to the E/C oligonucleotide is not surprising since this oligo contains the E-box motif which is a known binding site for b-HLH proteins and similar b-HLH regions have been identified in apoB present on VLDL and LDL.

The affinity for the HCMV IE promoter is not immediately obvious since careful analysis does not reveal an exact copy of an SRE, ISRE, FAS, or E/C sequence, however, the HCMV IE promotor region contains regulatory elements that are generally recognized by a large number of eukaryotic DNA-binding proteins, including a variety of different families of transcription factors, and it may therefore be possible that the identified b-HLH regions of apoB possess similar DNA binding properties.

Another possibility is that other yet unidentified regions of apoB are involved in the binding to the HCMV IE promoter region. The fact that HDL in contrast to VLDL and LDL do not bind to any of the oligos tested suggests that the DNA binding results from the specific interaction with apo B. These data support the hypothesis that apo B contains DNA binding domains which show homology with the DNA binding domains of SREBP-1, SREBP-2, ADD-1, and ISGF3γ and that apo B containing lipoproteins therefore bind to specific nucleotide sequences similar to those bound by these known DNA binding proteins.

Recent reports suggest a possible causal relationship between human cytomegalovirus (HCMV) and the development of atherosclerosis in humans. These reports together with data presented herein, which show that human LDL binds strongly to HCMV IE promotor sequences, led the inventors to investigate whether plasma LDL may play a role in the pathogenesis of HCMV induced atherosclerosis.

HCMV DNA sequences in the purified plasma LDL fraction of human subjects who tested seropositive for HCMV by polymerase chain reaction (PCR) were studied. The results of these studies show that a PCR product of the expected size (170 bp) could be detected with both primer sets (MTR2 and IE) in the purified plasma LDL fraction of HCMV seropositive subjects. However, this 170 bp DNA fragment could not be detected in the plasma samples of these subjects (lanes 6-8). These data suggest that the use of purified plasma LDL fractions for detection of CMV nucleic acid sequences by PCR techniques is more sensitive than when whole plasma samples are used. Furthermore, the increased PCR product yield from the purified plasma LDL fractions suggest strongly that HCMV DNA is predominantly associated with LDL within the plasma pool of HCMV seropositive subjects.

Example 4. Low-density Lipoprotein as a Natural Gene Transfer Vector. The discovery of the nucleic acid-binding properties apo B-100 suggested that lipoproteins containing apoB100, as naturally occurring liposomes, may function as gene transfer agents. By using highly purified low-density lipoprotein as such an agent, the inventors were able to transfect cultured human skin fibroblasts in vitro and to express a green fluorescent protein reporter gene in vivo. The gene transfer mediated by low-density lipoprotein was more efficient that that mediated by LipoFectin. Low-density lipoprotein also did not exhibit any toxicity, immunogenicity, or serum inhibition.

DNA-binding. In the Examples above, it was shown that highly purified human LDL binds to nucleic acids in a specific fashion. In order to establish whether rat lipoproteins can bind nucleic acids in a similar fashion, DNA-binding studies with different rat lipoprotein fractions were performed. A gel shift assay of linearized pBluescript KS and pBKCMV plasmid DNA and purified rat VLDL, LDL, and HDL fractions was performed. The data demonstrate clearly that the binding of nucleic acids is specific to the purified LDL fraction.

The binding of LDL to DNA is exhibited by the retarded electrophoretic migration of DNA in agarose gel that is caused by the formation of complexes of higher molecular weight. In contrast, purified fractions of VLDL and HDL did not bind any of the DNA samples tested. The fact that purified HDL did not bind DNA was expected, since endogenous HDL does not contain apo B-100. Surprisingly, there was no apparent binding of DNA to apo B-100-containing VLDL. It is possible that the DNA-binding assay, which employs ethidium bromide staining to detect DNA, lacks sensitivity or that VLDL does not bind to DNA under the conditions of the DNA-binding assay. Another explanation could be a difference in the conformation of apo B-100 present on LDL as opposed to VLDL because of a difference in the lipid composition and protein content of the two lipoprotein fractions.

In vitro Cell Transfection Studies. Based on the findings of the DNA-binding assay, transfection studies were performed using a prebound complex of LDL and plasmid DNA that contained a reporter gene that encodes GFP.

The data generated illustrated the successful transfection of how human skin fibroblasts with LDL and pEGFP-N1 plasmid DNA. The transfection process was monitored by expression of the GFP encoding gene and is driven by the HCMV IE promoter. In addition to fluorescent microscopic analysis, expression of GFP was confirmed by a qualitative ELISA using a primary antibody against recombinant GFP and an HRP-conjugated secondary antibody with .sigma.-phenylenediarnine as a chromogenic substrate.

Human skin fibroblasts transfected with LDL exhibited a significantly lower intensity of green fluorescence than did cells transfected with LipoFectin, indicating that the level of GFP expression was lower in these LDL-transfected cells. When the percentage of positively transfected cells were compared, however, transfection with LDL yielded a higher percentage of transfected cells than did transfection with LipoFectin (20 to 30% and 60 to 70%, respectively). In addition, LipoFectin-mediated transfection resulted in green fluorescence in the cell cytoplasm and in the nuclei, whereas LDL-mediated transfection resulted in green fluorescence predominantly in the cytoplasm.

Transfection assays in which LDL concentrations were as high as 250 g/ml of LDL protein produced no detectable effects on the confluence and viability of the cell cultures, whereas LipoFectin concentrations of 20 g/ml resulted in significant loss of cell viability. Control cells that were transfected with linearized pEGFP-NI plasmid DNA only exhibited no fluorescence.

In vivo Reporter Gene Expression. To evaluate whether LDL could be used as a vehicle for in vivo gene delivery, a prebound rat LDL-pEGFP-N1 complex was administered to 2-month-old female Sprague-Dawley rats. Cryosections of the liver and heart tissues of the treated animals that had been excised 2 days after the LDL-pEGFP-N1 complex showed significant levels of green fluorescence indicative of EGFP expression as determined by fluorescent microscopy.

The expression of GFP in the different tissues was confirmed by a qualitative ELISA using a primary antibody against recombinant GFP and an HRP-conjugated secondary. Antibody with σ-phenylenediamine as a chromogenic substrate. In contrast, only low levels of autofluorescence were observed in the cryosectioned tissues obtained from the control animals treated solely with linearized pEGFP-N1 DNA. These data demonstrate that purified LDL can be used in a prebound complex with DNA as an in vivo gene delivery system.

LDL Vaccine. This present invention is directed to compositions and methods for vaccination using gene delivery via a lipoprotein vector. More particularly, the present invention provides compositions and methods for the delivery of DNA vaccines using the low-density lipoprotein (LDL) vector. The vaccines include highly purified LDL complexed with a nucleic acid, e.g., DNA or RNA, encoding an antigen. Examples of antigens include tumor-associated protein antigens, or chimeras thereof, for application in cancer; viral antigens, e.g., genes encoding a viral-constituent protein antigens, or chimeras thereof; bacterial antigens, e.g., bacterial-constituent protein antigens, or chimeras thereof, or fungal antigen, e.g., genes encoding a fungal-constituent protein antigen, or a chimera thereof. The nucleic acid itself may be used as the antigen.

The LDL/nucleic acid vaccine may be administered by various routes including topically on the skin (dermal application), subcutaneously, intradermally, intramuscularly, intravenously, nasally or pharyngeally via nebulizer or spray, orally, rectally, or directly into an organ. The LDL/nucleic acid vaccine may be administered to induce a host normal cell to express a tumor-associated protein antigen, or a chimera thereof, for application in cancer, to express a viral-constituent protein antigen, or a chimera thereof, to express a bacterial-constituent protein antigen, or a chimera thereof, or to express a fungal-constituent protein antigen, or a chimera thereof.

Vaccines are used to elicit and establish immunity in an individual against an infectious pathogenic organism such as a virus, bacteria, fungus or a parasite. A vaccination or inoculation with a “killed” or “attenuated” version of an infectious entity or a component antigen such as a protein can confer lifelong immunity. Advances in molecular biology, specifically in the area of gene structure and methods of manipulation and transfer, along with the plethora of knowledge in areas of infectious diseases, including the biology, genetics and pathogenicity of viruses, bacterial, fungi, and parasites have lead to new methods for vaccination. The advances in recombinant DNA technology and their application in gene therapy have lead to the use of DNA as vaccines that are safer to use and easier to apply. However, of major concern has been the lack of an efficient and safe delivery system.

In DNA vaccination, a gene or gene fragment coding for an antigen protein is introduced to the individual's system, usually skeletal muscle cells, that then express, produce, the antigen protein that elicits or activates an immune response. Various approaches have been used to delivery the DNA vaccine including topical application of naked DNA, direct injection to the muscle, intravenous injection, etc. The DNA vaccine has been incorporated in liposome vectors, bacterial vectors, and viral vectors, all with limited success. The present application describes the use of the LDL vector as the delivery system for DNA vaccines. This vector can also be employed in the delivery of short interfering RNAs (siRNA) and microRNAs (miRNA). siRNA is thought to be an antiviral defense mechanism that regulates levels of RNA by cleaving complementary mRNA targets while miRNA is believed to attenuate translation of the mRNA.

Low-density lipoprotein, LDL, has been shown to have the capacity to bind nucleic acids, DNA and RNA, and to transfect cells including the delivery of DNA to the cell's nucleus. This capacity is imparted by the major apolipoprotein, apo B-100, that characterizes all low-density lipoproteins including very low-density lipoprotein (VLDL), intermediate-density lipoprotein, and lipoprotein [a]. LDL uptake by cells is mediated by the LDL receptor that recognizes and binds to a specific ligand region on the apo B-100 molecule. Binding to nucleic acid by LDL is based on the presence of regions in the apo B-100 primary structure that are similar to the DNA-binding domains of the transcription factors, ISGF3γ (IRF9), STATs, IRFs, and SREBPs as well as by the presence of RNA-binding lysine homology (KH) motifs of hnRNPs and RNPs. Translocation of DNA to the cell nucleus is facilitated by the presence of numerous bipartite nuclear localization sequences (NLS) in the apo B-100 sequence.

Binding to nucleic acids. A modified gel shift assay was used to show binding of highly purified preparations of human LDLs to fragmented genomic DNA, plasmid DNA, synthetic oligonucleotides, and total RNA from human liver. Purified LDL was shown to bind synthetic oligomers that contain the ISRE (5′-GGGAAACCGAAACTG) and E/C (5′-CANNTG, E-box motif and CCAAT, adipocyte-specific genes promoter site). Lower binding was observed for synthetic oligonucleotides of the FAS element (5′-GTCCAATTGGTC) that also contains overlapping E-box motifs and C element. Point mutations introduced into oligonucleotides resulted in a decrease in binding of LDL to the ISRE and an increase in LDL binding to the SRE. Binding intensities of these oligonucleotides to very-low-density lipoprotein (VLDL) were determined to be less than 1% of total labeled DNA. LDL was also observed to bind preferentially to plasmid DNA containing the hCMV IE2 promoter region. In studies using human liver total RNA, RNA for five different genes was recovered from LDL and VLDL bands. The corresponding DNA was produced by reverse transcriptase—polymerase chain reaction (rt-PCR) and then subjected to sequence analysis. DNA isolated with VLDL contains CAAT and TATA motifs and appear to be related to 5′ flanking regions of unidentified genes. No matches for the sequences of genes isolated with LDL were found.

In vitro nuclear translocation and protein expression. Cell uptake and translocation of plasmid DNA to the cell nucleus using the LDL vector was first demonstrated in human fibroblast cells. Uptake of dye-labeled DNA was seen to occur rapidly; about 100% of cells incorporated the LDL/DNA complex within 10 minutes of exposure. Translocation of dye-labeled plasmid DNA to the fibroblast nucleus as observed as early as 20 seconds after exposure to the LDL/DNA complex in some cells. Expression of functional protein was also demonstrated in human fibroblast cells using a plasmid containing the green fluorescent protein (GFP) gene. Fluorescence by the GFP was observed in a few fibroblast cells as early as 30 minutes after exposure to the LDL/DNA plasmid complex.

In vivo transfection of the rat. Expression of the reporter GFP was also demonstrated in several tissues of the rat including the heart, lung, liver and spinal cord. Highly purified rat LDL combined with linearized GFP plasmid DNA was injected via several routes to two-month old female rats. Fluorescent microscopy of frozen tissue sections revealed the expression of GFP.

Materials and methods. Isolation of plasma lipoproteins. Human plasma LDL was obtained from Chao-Yuh Yang, Ph.D., Associate Professor, Division of Atherosclerosis and Lipoprotein Research, Department of Medicine, Baylor College of Medicine, Houston, Tex. in phosphate-buffered saline (pH 7.4) with EDTA.

Plasmid DNA vaccine. The plasmid DNA vaccine was obtained from Mithragen, Inc. The plasmid, pRc/CMV2, was obtained from Invitrogen, Inc. Bacillus anthracis capsule biosynthesis gene, capB, or the Bacillus anthracis gene associated with depolymerization of the capsule, dep were inserted at the Hind III site

Non-covalent labeling of plasmid DNA with BoBo1. Three microliters of Bobo1-iodide™, obtained as a 1 mM solution in DMSO from Molecular Probes, was added to 10 μg of DNA at a concentration of 0.5 μg/μl in phosphate buffered saline and the solution was incubated at ambient temperature for 1 h.

In vitro cell transfection studies. Several cell types, NIH 3T3 cells (a mouse cell line), MCF7 cells (human breast cancer cell line), HEP1 cells (human hepatocytes), 38 lung cells (human lung cells), LNCaP cells (human lymph node prostate cancer cell line), SK-N-BE2 human neuroblastoma cells, and SK37 human melanoma cells, were used to test the transfect efficacy using the LDL vector as carrier of the BoBo1-labeled plasmid DNA. Cells were cultured in the media described in Table 1. Cells were grown on polylysine-coated cover slips in 1.0 ml wells containing RPMI supplemented with 10% FCS, and required co-factors (MCF7, 38 Lung, and LNCaP) or in DMEM with 10% FCS and required co-factors (all others) at 37° in an atmosphere of 5% CO₂ in a humidified incubator. After reaching 60-70% confluence, the media was removed and cells were washed thrice with phosphate-buffered saline then incubated for 4 hours in complete medium minus FCS. The cover slips were then recovered from each well and placed inverted over a well of a hanging drop slides containing complete medium less FCS plus the LDL combined with the plasmid dye-labeled DNA vaccine or a well containing medium less FCS plus naked plasmid dye-labeled DNA vaccine.

Briefly, the plasmid dye-labeled DNA vaccine included a commercially obtained plasmid, pRc/CMV2, inserted with the Bacillus anthracis capsule biosynthesis gene, capB, or the Bacillus anthracis gene associated with depolymerization of the capsule, dep. These plasmid vaccines were provided by Mithragen, Inc. and were used to demonstrate the utility and transfection efficacy of the LDL vector. Ten μg of highly purified human LDL was combined with 1.0 μg of BoBo1-labeled plasmid DNA and allowed to stand at ambient temperature for a minimum of 15 minutes before adding to the complete media less FCS. Cell uptake was then observed using fluorescent microscopy.

TABLE 1
Cells and Culture Media.

| Cell Line | Medium |
|---|---|
| Hep1 (liver) liver; ascites; adenocarcinoma | DMEM with 10% FBS, 100 units Penicillin G |
| The SK-HEP-1 line has been identified as | Sodium, 100 units/ml Streptomycin Sulfate and |
| being of endothelial origin. | 250 ng/ml Amphotericin B. |
| Hela (cervical) cervix; epithelial; | DMEM with 10% FBS, 100 units Penicillin G |
| adenocarcinoma | Sodium, 100 units/ml Streptomycin Sulfate and |
| | 250 ng/ml Amphotericin B. |
| MCF7 (breast) mammary gland; breast; | RPMI 1640 with 10% FBS, 100 units Penicillin |
| epithelial; metastatic site: pleural effusion | G Sodium, 100 units/ml Streptomycin Sulfate |
| adenocarcinoma | and 250 ng/ml Amphotericin B. |
| HT-29 (colon) human; colorectal | DMEM with 10% FBS, 100 units Penicillin G |
| adenocarcinoma | Sodium, 100 units/ml Streptomycin Sulfate and |
| | 250 ng/ml Amphotericin B. |
| SK37 (melanoma) | DMEM with 10% FBS, 100 units Penicillin G |
| Could not find anything on this cell line | Sodium, 100 units/ml Streptomycin Sulfate and |
| At ATCC. | 250 ng/ml Amphotericin B. |
| 293 (kidney epithelial) human kidney; | DMEM with 10% FBS, 100 units Penicillin G |
| transformed with adenovirus 5 DNA | Sodium, 100 units/ml Strepmycin Sulfate and |
| | 250 ng/ml Amphotericin B. |
| LNCaP (prostate) established from a metastatic | RPMI 1640 with 10% FBS, 100 units Penicillin |
| lesion of human prostatic adenocarcinoma in | G Sodium, 100 units/ml Streptomycin Sulfate |
| the lymphnode. metastatic site: left | and 250 ng/ml Amphotericin B. |
| supraclavicular lymph node carcinoma | |
| 38 (lung) | DMEM with 10% FBS, 100 units Penicillin G |
| lung; fibroblast; normal | Sodium, 100 units/ml Streptomycin Sulfate and |
| | 250 ng/ml Amphotericin B. |
| SKNBE12 (neuroblastoma) or | DMEM with 10% FBS, 100 units Penicillin G |
| SK-N-BE(2) | Sodium, 100 units/ml Streptomycin Sulfate and |
| brain; metastatic site: bone marrow | 250 ng/ml Amphotericin B. |
| neuroblastoma | |
| NIH3T3 (mouse fibroblast) embryo The | DMEM with 10% FBS, 100 units Penicillin G |
| NIH/3T3, a continuous cell line of highly | Sodium, 100 units/ml Streptomycin Sulfate and |
| contact-inhibited cells was established from | 250 ng/ml Amphotericin B. |
| NIH Swiss mouse embryo cultures in the same | |
| manner as the original random bred 3T3 | |
| (ATCC CCL-92) and the inbred BALB/c 3T3 | |
| (ATCC CCL-163). | |

Fluorescent microscopy. Microscopy was performed by using an Olympus Model BH-2 fluorescent microscope (Olympus, USA). The filter set used included the following filter cubes, a fluorescein (FITC/TRITC) #51004V2 cube, a Chroma HQ-GFP NB710 cube #41020 (Chroma Technology, Brattleboro, Vt., USA, and an Olympus dichroicDM500 (BP490) filter. Transfection efficiency was assessed visually by examination of transfected cells in changing fields of view.

Cell transfection studies. Cell uptake of the anthrax gene plasmid combined with LDL was demonstrated in several different cell types including, NIH 3T3 cells (a mouse cell line), MCF7 cells (human breast cancer cell line), HEP1 cells (human hepatocytes), 38 lung cells (human lung cells), LNCaP cells (human lymph node prostate cancer cell line), HeLa (cervical cancer), HT-29 (colon cancer), SK-N-BE2 human neuroblastoma cells, SK37 human melanoma cells, and 293 (human kidney epithelial cells) as summarized in Table 2.

TABLE 2
Cell Type and Transfection Efficiency.

| | LDL/DNA | DNA Only |
|---|---|---|
| Cell type | % cells transfected | Control |
| NIH 3T3 (mouse) | 80-100% | None |
| MCF 7 | 80-100% | None |
| Hep 1 | 80-100% | None |
| SK-N-BE2 (neuroblastoma) | >80% | None |
| 38 (lung) | ˜50% | None |
| LinCap (prostate) | ˜50% | None |
| SK37 (melanoma) | ˜50% | None |
| HEK 293 (kidney) | ˜5% | None |
| Dendritic | 0% | None |
| Macrophages | 0% | None |

Knowledge of the functional domains of the apo B-100 molecule is limited, largely because the focus of research has been on the role of this massive molecule in the transport and delivery of lipids in plasma. Although a role for LDL as a biomodulator of the immune response and lymphocyte function was inferred earlier (52-54), only a few reports exist ascribing function to specific regions of apo B-100 (12, 13, 27, 55-59). B-100 domains that contain basic, positively charged amino acids impart positive charge on the surface of the LDL particle giving it the capacity to interact with negatively charged moieties such as sialylated glycoproteins. In the past, this type of ionic interaction has been considered nonspecific and unimportant as well as a nuisance in the purification of LDLs. As disclosed herein, several regions in the apo B-100 sequence are characterized by amino acid motifs that are functional regions in DNA binding domains of transcription factors. These apo B-100 regions may also impart human LDL with the capacity to bind plasmid DNA vaccines. Further, the presence of eleven KH motifs in the apo B-100 molecule lends LDL RNA binding capability. The numerous nuclear localization sequences, NLS, also present in the apo B-100 may be part of a nuclear translocation mechanism and immune biomodulator system that is still undiscovered in cells.

The present inventor had established previously that LDL a natural gene delivery vector. LDL was purified and used with pEGFP-N1, a commercial plasmid that includes the human cytomegalovirus immediate early 2 promoter and a reporter gene, to transfect fibroblast cells in vitro. LDL has been used as a gene delivery vector previously (29-31) because of its receptor specificity and natural liposome qualities. In was found that apo B-100 binds to the IE2 promoter (it does not bind to the plasmid minus IE2, data not shown), the LDL/DNA complex enters the cell via the B/E receptor or through other mechanisms such as membrane fusion, and as a whole or in part translocates the DNA to the nucleus. The mechanism(s) by which the LDL-DNA complex exits the endosome is not known. The delivery system was demonstrated in human fibroblast cells in culture and in the rat in vivo. The current results demonstrate that LDL functions as a gene delivery vector in the plasma beyond mere lipid transport. Therefore, the LDL gene delivery vector of the present invention may be used in a wide variety of applications to transfect DNA vectors for DNA vaccines.

The present invention may be used in conjunction with immunological techniques that are well-known in the art, e.g., U.S. Pat. No. 6,602,510, relevant techniques, sequences, assays and methods incorporated herein by reference, which teaches the methods for selecting, evaluating and constructing genes for vaccine production against tumor associated antigen peptides and vaccines for presentation by class I MHC (human HLA). For example, the present invention may be used in conjunction with minigenes, constructs and peptide sequences that may be used deliver a gene construct that encodes one or more peptides that are presented on APCs by class I MHC in the context of tumor vaccines. Furthermore, basic techniques for stimulation of CTL and HTL responses, determining the binding affinity of peptide epitopes for HLA molecules, peptide epitope binding motifs and supermotifs, HLA-A2 supermotifs, HLA-A2.1 motifs, HLA class II motifs and PADRE™, enhancing population coverage of the vaccine, immune response-stimulating peptide epitope analogs, preparation of peptide epitopes, assays to detect T-cell responses, uses for peptide epitopes for evaluating immune responses, vaccine compositions, minigene vaccines, combinations of CTL peptides with helper peptides, combinations of CTL peptides with T cell priming materials, vaccine compositions including dendritic cells pulsed with CTL and/or HTL epitopes, administration of vaccines for therapeutic or prophylactic purposes and kits. Epitopes presented by Class II MHC, techniques, sequences, assays and methods for identifying useful vaccine epitopes are taught by, e.g., U.S. Pat. No. 6,689,363, relevant portions incorporated herein by reference. When used in conjunction with the present invention, the peptide epitopes may be included alone, as concatamers, as fusion proteins and the like on the nucleic acid vector for delivery by LDL.

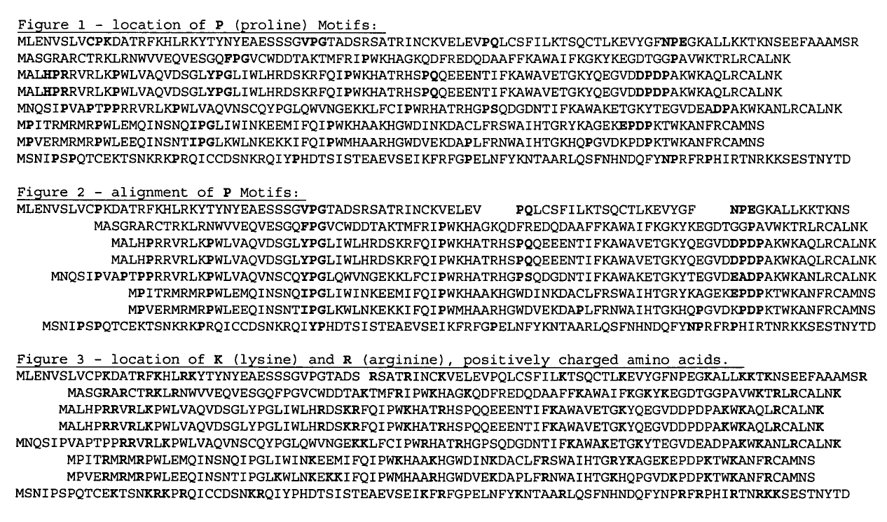

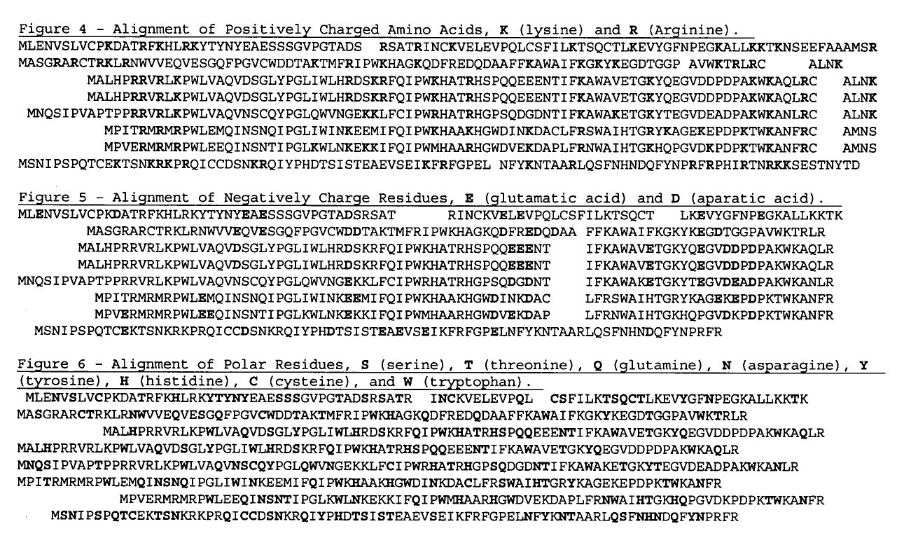

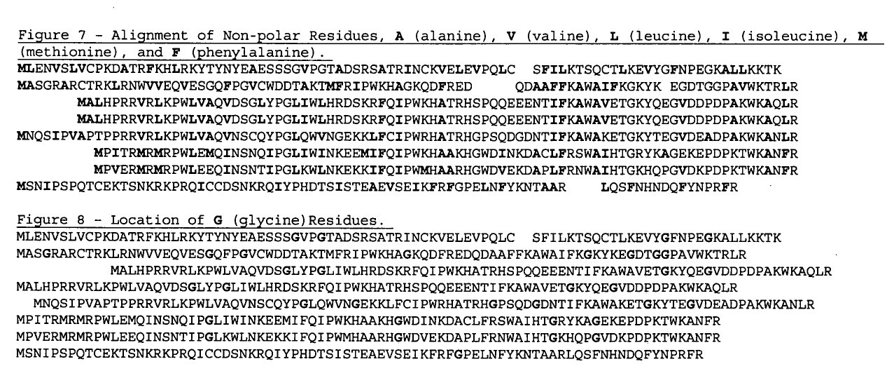

FIG. 1-8 show a sequence comparison of DNA-binding domains to region in apo b.

Sequence alignment of the amino ends of apo B-100, Interferon Regulatory Factor Family (IRF) proteins, and H86 Herpes Virus protein is presented. The sequence of apo B-100 is the top sequence, IRF9 (ISGF3γ) is second, and the next five are IRF proteins that are most like apo B-100, and the last sequence is of H86 a Herpes virus protein, also a transactivation protein.

The following logic was used in identifying a DNA-binding Domain in the amino terminus of the apo B-100 sequence.

1. The location of Proline and the types of amino acids adjacent to it is an important consideration when trying to relate the sequence to actual structure. Proline residues provide a degree conformational rigidity useful in locating certain structural motifs in proteins. The Proline motifs were first located in the amino termini of the apo B-100, IRF and H86 viral protein sequences by highlighting in bold font the letter P (Proline) and amino acids before and after P, for example, PK, PR, PG, PQ, NPE and DPE. Next, matching the motifs aligned the sequences.

2. Similarly, clusters of the other groups of amino acids were located, for example, positively charged amino acids K (lysine) and R (arginine). These clusters are common in DNA-binding domains.

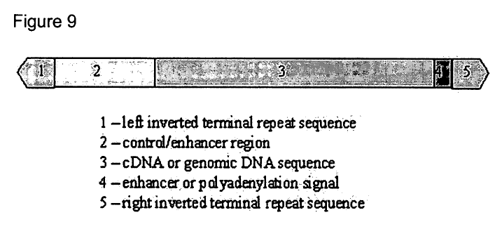

FIG. 9 is a generalized expression construct for use with the present invention. The construct may include five regions: (1) a left inverted terminal repeat sequence; (2) a control/enhancer region; (3) a cDNA or genomic DNA sequence or insert that expressed an antigen, antigen presenting protein, T or B cell receptor or other immune enhancer (lymphokines and the like) or even two or more combinations thereof; (4) an enhancer or polyadenylation sequence; and (5) a right inverted terminal repeat. As will be apparent to those of skill in the art, any number of terminal repeats, enhancers, promoters, start-sites (e.g., consensus Kozak regions); polyadenylation sequences may be assembled in operative order to maximize message and or protein expression.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### References

1. Kannel, W. B., W. P., Castelli, T. Gordon, and P. M. McNamara. 1971. Serum cholesterol, lipoproteins, and the risk of coronary heart disease. The Framingham Study. Ann. Intern. Med. 74: 1-12.
2. Breslow, J. L. 1988. Apolipoprotein and genetic variation and human disease. Physiol. Rev. 68: 85-132.
3. Cambien, F., J.-M. Warnet, A. Jacqueson, P. Ducimetiere, J.-L. Richard, and J.-R. Claude. 1987. Relation of parental history of early myocardial infarction to the level of apoprotein B in men. Circulation 76: 266-271.
4. Powell, L. M., S. C. Wallis, R. J. Pease, Y. H. Edwards, T. J. Knott, and J. Scott. 1987. A novel form of tissue-specific RNA processing produces apolipoprotein B-48 in intestine. Cell 50: 831-840.
5. Chen, S.-H., G. Habib, C.-Y. Yang, Z.-W. Gu, G. R. Lee, S.-A. Weng, S. R. Silberman, S.-J. Cai, J. P. Deslypere, M. Rosseneu, A. M. Gotto, Jr., W.-H. Li, and L. Chan. 1987. Apolipoprotein B-48 is the product of a messenger RNA with an organ-specific in-frame stop codon. Science 238: 363-366.
6. Scott, J. 1989. The molecular and cell biology of apolipoprotein B. Mol. Biol. Med. 6: 65-80.
7. Chen, S. H., C. Y., Yang, P. F. Chen, D. Setzer, M. Tanimura, W. H. Li, A. M. Gotto, Jr., and L. Chan. 1986. The complete cDNA and amino acid sequence of human apolipoprotein B-100. J. Biol. Chem. 261: 12918-12921.
8. Law, S. W., S. M. Grant, K. Higuchi, A. Hospattankar, K. Lackner, N. Lu, and H. B. Brewer, Jr. 1986. Human liver apolipoprotein B-100 cDNA: Complete nucleic acid and derived amino acid sequence. Proc. Natl. Acad. Sci. USA 83: 8142-8146.
9. Yang, C. Y., S.-H. Chen, S. H. Gianturco, W. A. Bradley, J. T. Sparrow, M. Tanimura, W. H. Li, D. A. Sparrow, H. DeLoof, M. Rosseneu, F. S. Lee, Z. W. Gu, A. M. Gotto, Jr., and L. Chan. 1986. Sequence, structure, receptor-binding domains and internal repeats of human apolipoprotein B-100. Nature 323: 738-742.
10. Knott, T. J., R. J. Pease, L. M. Powell, S. C. Wallis, S. C. Rall, Jr., T. L. Innerarity, B. Blackhart, W. H. Taylor, Y. L. Marcel, R. Milne, D. Johnson, M. Fuller, A. J. Lusis, B. J. McCarthy, R. W. Mahley, B. Levy-Wilson, and J. Scott. 1986. Complete protein sequence and identification of structural domains of human apolipoprotein B. Nature 323: 734-738.
11. Cladaras, C., M. Hadzopoulou-Cladaras, R. T. Nolte, D. Atkinson, and V. I. Zannis. 1986. The complete sequence and structural analysis of human apolipoprotein B-100: Relationship between apo B-100 and apo B-48 forms. EMBO J. 5: 3495-3507.
12. Guevara, J., Jr., N. V. Valentinova, O. Garcia, A. M. Gotto, C.-Y. Yang, S. Legal, J. Gaubatz, and J. T. Sparrow. 1996. Interaction of apolipoprotein[a] with apolipoprotein B-100 Cys³⁷³⁴ region in lipoprotein[a] is confirmed immunochemically. J. Prot. Chem. 15: 17-25.
13. Weisgraber, K. H. and S. C. Rall, Jr. 1987. Human apolipoprotein B-100 heparin binding-sites. J. Biol. Chem. 262: 11097-11103.
14. Scott-Bruden, T., T. J. Resink, A. W. A. Hahn, U. Baur, R. J. Box, and F. R. Buhler. 1989. Induction of growth-related metabolism in human vascular smooth muscle cells by low density lipoprotein. J. Biol. Chem. 264: 12582-12589.
15. Smimov, V. N., T. A. Voyno-Yasenetskaya, A. S. Antonov, M. E. Lukashev, V. P. Shirinsky, V. V. Tertov, and V. A. Tkachuk. 1990. Vascular sinal tansduction and atherosclerosis. Annals New York Academy of Sciences 598: 167-181.
16. Jackson, T. K., A. I. Salhanick, J. Elovson, M. L. Deichman, and J. M. Amatruda. 1990. Insulin regulates apolipoprotein B turnover and phosphorylation in rat hepatocytes. J. Clin. Invest. 86: 1746-1751.
17. Sachinidis, A., R. Locher, and W. Vetter. 1991. Generation of intracellular signals by low density lipoprotein is independent of the classical LDL receptor. Am. J. Hypertension 4: 274-279.
18. Voyno-Yasenetskaya, T., L. G. Dobbs, S. K. Erickson, and R. L. Hamilton. 1993. Low density lipoprotein- and high density lipoprotein-mediated signal transduction and exocytosis in alveolar type II cells. Proc. Natl. Acad. Sci. USA 90: 4256-4260.
19. Guevara, J., Jr., J. T. Sparrow, N. V. Valentinova, D. Davison, and J. D. Morrisett. 1994. Src signal sequences in apoprotein B-100: Does LDL or Lp[a] play a role in signal transduction. In Multiple Risk Factors in Cardiovascular Disease. A. Yamamoto, editor. Churchill Livingstone, Japan K. K. 141-145.
20. Bochkov, V. N., V. A. Tkachuk, Y. S. Kuzmenko, Y. L. Borisova, F. R. Buhler, and T. J. Resink. 1994. Characteristics of low and high density lipoprotein binding and lipoprotein-induced signaling in quiescent human vascular smooth muscle cells. Molecular Pharmacology 45: 262-270.
21. Resink, T. J., V. Tybin, J. Bernhardt, S. Orlov, F. R. Buhler, and V. A. Tkachuk. 1993. Cellular signalling by lipoproteins in cultured smooth muscle cells from spontaneously hypertensive rats. J. Vascular Research 30: 169-180.
22. Bochkov, V. N., V. A. Tkachuk, A. W. A. Hahn, J. Bernhardt, F. R. Buhler, and T. J. Resink. 1993. Concerted efffects of lipoproteins and angiotensin II on signal transduction processes in vascular smooth muscle cells. Arterioscler. Thromb. 13: 1261-1269.
23. Drobnik, W., C. Mollers, T. J. Resink, and G. Schmitz. 1995. Activation of phospatidylinositol-specific phospholipase C in response to HDL₃ and LDL is markedly reduced in cultured fibroblasts from Tangier patients. Arterioscler. Thromb. Vascular Biol. 15: 1369-1377.
24. Tensen, C. P., E. R. van Kesteren, R. J. Planta, K. J. A. Cox, J. F. Burke, H. van Heerikhuizen, and E. Vreugdenhil. 1994. A G protein-coupled receptor with low density lipoprotein-binding motifs suggests a role for lipoproteins in G-lined signal transduction. Proc. Natl. Acad. Sci. USA 91: 4816-4820.
25. Tkachuk, V. A., Y. S. Kuzmenko, T. J. Resink, D. V. Stambolsky, and V. N. Bochkov. 1994. Atypical low density lipoprotein binding site that may medicate lipoprotein-induced signal transduction. Molecular Pahrmacology 46: 1129-1137.
26. Mollers, C., W. Drobnik, T. J. Resink, and G. Schmitz. High-density lipoprotein and low-density lipoprotein-mediated signal transduction in cultured human skin fibroblasts. Cellular Signalling 7: 695-707.
27. Guevara, J., Jr., E. T. Walch, H. F. Epstein, J. T. Sparrow, A. M. Gotto, and N. V. Valentinova. 1995. Evidence that apo B-100 of low-density lipoproteins is a novel Src-related protein kinase. J. Protein Chem. 14: 627-631.
28. Firestone, R. A. 1994. Low-density lipoprotein as a vehicle for targeting antitumor compounds to cancer cells. Bioconjugate Chem. 5: 105-113.
29. Kim, J-S., A. Maruyama, T. Akaike, and S. W. Kim. 1995. Terplex system of lipoprotein, cationic polymer, and DNA gene delivery. Pharm. Res. 12(9): S80.
30. Kim, J-S., A. Maruyama, T. Akaike, and S. W. Kim. 1998. Terplex DNA delivery system as a gene carrier. Pharm. Res. 15(1): 116-121.
31. Kim, J-S., B. I. Kim, A. Maruyama, T. Akaike, and S. W. Kim. 1998. A new non-viral DNA delivery vector: the terplex system. J. Controlled Release 53: 175-182.
32. Hara, T., Y. Tan, and L. Huang. 1997. In vivo gene delivery to the liver using reconstituted chylomicron remnants as a novel nonviral vector. Proc. Nat'l Acad. Sci. USA 94: 14547-14552.
33. Desjarlais, J. R., and J. M. Berg. 1994. Length-encoded multiplex binding site determination: application to zinc finger proteins. Proc. Natl. Acad. Sci. USA 91: 110099-11103.
34. Gibson, T. J., J. D. Thompson, and R. A. Abagyan. 1993. Proposed structure for the DNA-binding domain of the helix-loop-helix family of eukaryotic gene regulatory proteins. Protein Engineering 6: 41-50.
35. Tontonoz, P., J. B. Kim, R. A. Graves, and B. M. Spiegelman. 1993. ADD1: A novel helix-loop-helix transcription factor associated with adipocyte determination and differentiation. Mol. Cell. Biol. 13: 4753-4759.
36. Veals, S. A., C. Schindler, D. Leonard, X.-Y. Fu, R. Aebersold, J. E. Darnell, Jr., and D. E. Levy. 1992. Subunit of an alpha-interferon-responsive transcription factor is related to interferon regulatory factor and Myb families of DNA-binding proteins. Mol. Cell. Biol. 12: 3315-3324.
37. Escalante, C. R., J. Yie, D. Thanos, and A. K. Aggarwal. 1998. Structure of IRF-1 with bound DNA reveals determinants of interferon regulation. Nature 391: 103-106.
38. Boulanger, P. A., S. K. Yoshinaga, and A. J. Berk. 1987. DNA-binding properties and characterization of human transcription factor TFIIIC2. J Biol Chem 262(31): 15098-15105.
39. Schumaker, V. N., and D. L. Puppione. 1986. Sequential flotation ultracentrifugation. Methods Enzymol. 128: 155-170.
40. Lowry, O. H., N. J. Rosebrough, A. L. Farr, and R. J. Randall. 1951. Protein measurement with the Folin phenol reagent. J. Biol. Chem. 193: 265-275.
41. Robbins, J., S. M. Dilworth, R. A. Laskey, and C. Dingwall. 1991. Two interdependent basic domains in nucleoplasmin nuclear targeting sequence: Identification of a class of bipartite nuclear targeting sequence. Cell 64: 615-623. 42. ; Dingwall, C., and R. A. Laskey. 1991. Nuclear targeting sequences—a consensus? Trends Biochem. Sci. 16: 478-481.
43. Garcia-Bustos, J., J. Heitman, and M. N. Hall. 1991. Nuclear protein localization. Biochim. Biophy. Acta. 1071: 83-101.
44. Schlenstedt, G. 1996. Protein import into the nucleus. Fed. Euro. Biochem. Soc. 389: 75-79.
45. Williams, S. C., N. D. Angerer, and P. F. Johnson. 1997. C/EBP proteins contain nuclear localization signals imbedded in their basic regions. Gene Expr. 6: 371-385.
46. Horvath, C. M., Z. Wen, and J. E. Darnell, Jr. 1995. A STAT protein domain that determines DNA sequence recognition suggests a novel DNA-binding domain. Genes Dev. 9: 984-994.
47. Burd, C. G., and G. Dreyfuss. 1994. Conserved structures and diversity of functions of RNA-binding proteins. Science 265: 615-621.
48. Gibson, T. J., J. D. Thompson, and J. Heringa. 1993. The KH domain occurs in a diverse set of RNA-binding proteins that include the antiterminator NusA and is probably involved in binding to nucleic acid. Fed. Euro. Biochem. Soc. 324: 361-366.
49. Siomi, H., M. Choi, M. C. Siomi, R. L. Nussbaum, and G. Dreyfuss. 1994. Essential role for KH domains in RNA binding: Impaired RNA binding by a mutation in the KH domain of FMR1 that causes fragile X Syndrome. Cell 77: 33-39.
50. Ho, W. K. K., and K. F. Shortridge. Serum lipoproteins as inhibitors of haemagglutination by rubella virus. Lipids 12: 85-91, 1976.
51. Xu, X., Y-L. Sun, and T. Hoey. 1996. Cooperative DNA binding and sequence-selective recognition conferred by the STAT amino-terminal domain. Science 273: 794-797.
52. Shine, T. E., J. R. Little, V. Shore, G. Medoff, and A. Abegg. 1980. Effect of exogenous lipids and lipoproteins on the primary immune response in vitro. Eur. J. Immunol. 10: 714-718.
53. Edgington, T. S., and L. K. Curtiss. 1981. Plasma lipoproteins with bioregulatory properties including the capacity to regulate lymphocyte function and the immune response. Cancer Research 41: 3786-3788.
54. Macy, M., Y. Okano, A. D. Cardin, E. M. Avila, and J. A. K. Harmony. 1983. Suppression of lymphocyte activation by plasma lipoproteins. Cancer Research (Suppl.) 43: 2496s-2502s.
55. Cardin, A. D., R. L. Bowlin, and J. L. Krstenansky. 1988. Inhibition of lymphocyte proliferation by synthetic peptides homologous to human plasma apolipoproteins B and E. Biochem. Biophys. Res. Comm. 154: 741-745.
56. Innerarity, T. L., K. H. Weisgraber, S. C. Rall, Jr., and R. W. Mahley. 1986. Functional domains of apolipoprotein E and apolipoprotein B. Acta. Med. Scand. Suppl. 715: 51-59.
57. Hirose, N., D. T. Blankenship, M. A. Krivanek, R. L. Jackson, and A. D. Cardin. 1987. Isolation and characterization of four heparing-binding cyanogen bromide peptides of human plasma apolipoprotein B. Biochemistry 26: 5505-5512.
58. Milne, R., R. Theolis, Jr., R. Maurice, R. J. Pease, P. K. Weech, E. Rassart, J.-C. Fruchart, J. Scott, and Y. L. Marcel. J. Biol. Chem. 264: 19754-19760.
59. Brown, M. S., and J. L. Goldstein. 1986. A receptor-mediated pathway for cholesterol homeostasis. Science 232: 34-47.

1: A method for expressing an antigen in a human cell comprising: contacting a cell with a composition comprising a native low density lipoprotein and a nucleic acid comprising an expression cassette encoding the antigen bound to the low density lipoprotein; delivering the composition to the cell under conditions permitting transfer of the composition into the cell; and culturing the cell under conditions that permit the expression of the antigen.

2: The method of claim 1, wherein the antigen comprises a tumor rejection antigen precursor selected from BAGE, GAGE, MAGE and DAGE, a superantigen, an antigen that triggers T-cell anergy, an antigen that triggers a B-cell response, an antigen having T cell epitope and a B-cell epitope, an antigen from the group consisting of a pathogen, a virus, bacteria, fungus, protozoan, arthropod, nematode and helminth, an intracellular pathogen, Listeria monocytogenes, Salmonella typhimurium, Neisseria gonorrhoeae, Mycobacterium avium, Mycobacterium tuberculosis, Mycobacterium leprae, Brucella abortus, and Candida albicans, a malarial antigen, one or more genes or fusion protein chimeras selected from CSP-1, STARP, SALSA, SSP-2, LSA-1, EXP-1, LSA-3, RAP-1, RAP-2, SERA-1, MSP-1, MSP-2, MSP-3, MSP-4, MSP-5, AMA-1, EBA-175, Pf35, Pf55, RESA, EMP-1, GLURP, Pfs16, Pfs25, Pfs28, Pfs45, Pfs48, Pfs230, Pfg27, and Pfs28 and combinations thereof.

3-9 (canceled)

10: A composition comprising a native low density lipoprotein and a nucleic acid comprising an expression cassette encoding an antigen bound to the low density lipoprotein.

11: The composition of claim 10, wherein the antigen comprises a tumor rejection antigen precursor selected from BAGE, GAGE, MAGE and DAGE, a superantigen, an antigen that triggers T-cell anergy, an antigen that triggers a B-cell response, an antigen having T cell epitope and a B-cell epitope, an antigen from the goup consisting of a pathogen, a virus, bacteria, fungus, protozoan, arthropod, nematode and helminth, an intracellular pathogen, Listeria monocytogenes, Salmonella typhimurium, Neisseria gonorrhoeae, Mycobacterium avium, Mycobacterium tuberculosis, Mycobacterium leprae, Brucella abortus, and Candida albicans, a malarial antigen, one or more genes or fusion protein chimeras selected from CSP-1, STARP, SALSA, SSP-2, LSA-1, EXP-1, LSA-3, RAP-1, RAP-2, SERA-1, MSP-1, MSP-2, MSP-3, MSP-4, MSP-5, AMA-1, EBA-175, Pf35, Pf55, RESA, EMP-1, GLURP, Pfs16, Pfs25, Pfs28, Pfs45, Pfs48, Pfs230, Pfg27, and Pfs28 and combinations thereof.

12-20 (canceled)

21: A method of screening for an antigenic one open reading frame, comprising the steps of: preparing in vitro at least one linear or circular expression element comprising the open reading frame of an antigen linked to a promoter and a low density lipoprotein; and delivering the at least one linear or circular expression element into a cell within an animal without intervening cloning or bacterial propagation; and assaying the immune response of an animal by expression of the antigen encoded by the open reading frame in the expression element.

22: The method of claim 21, wherein the linear or circular expression element is injected into the animal.

23: The method of claim 21, wherein the linear or circular expression element further comprises a terminator linked to the open reading frame.

24: The method of claim 21, wherein the open reading frame is from a pathogen genomic sequence.

25: The method of claim 21, wherein preparing.the expression element comprises linking non-covalently the promoter to the open reading frame.

26: The method of claim 21, wherein preparation of the expression element comprises polymerase chain reaction.

27: The method of claim 21, wherein preparing the expression element comprises chemical synthesis of the open reading frame.

28: The method of claim 21, further comprising identifying an antibody produced by the animal and directed against the polypeptide encoded by the open reading frame.

29: The method of claim 24, wherein the pathogen is a virus, bacterium, fungus, algae, protozoan, arthropod, nematode, helminth, or plant.

30: The method of claim 24, further comprising testing an animal comprising the cell by challenge with the pathogen.

31: The method of claim 25, wherein preparing the expression element further comprises non-covalently linking a terminator to the open reading frame.

32: The method of claim 25, wherein the open reading frame is produced in vivo and then non-covalently linked to the promoter in vitro.

33: The method of claim 26, wherein preparation of the expression element comprises polymerase chain reaction to produce the open reading frame.

34: The method of claim 28, further comprising isolating the antibody.

35: The method of claim 30, wherein the animal is protected from the challenge with the pathogen.

36: The method of claim 25, further comprising identifying one or more antigens conferring protection to the animal.

37: A method for expressing an antigen in a mammalian cell comprising: contacting a cell with a composition comprising a native low density lipoprotein from the same mammal as the mammalian cell and a nucleic acid comprising an expression cassette encoding the antigen bound to the low density lipoprotein; delivering the composition to the cell under conditions permitting transfer of the composition into the cell; and culturing the cell under conditions that permit the expression of the antigen.


SEQUENCE LISTING (10 sequences)

SEQ ID NO: 1 - protein, 98 aa, Homo sapiens
MLENVSLVCPKDATRFKHLRKYTYNYEAESSSGVPGTADSRSATRINCKVELEVPQLCSFILKTSQCTLKEVYGFNPEGKALLKKTKNSEEFAAAMSR

SEQ ID NO: 2 - protein, 90 aa, Homo sapiens
MASGRARCTRKLRNWVVEQVESGQFPGVCWDDTAKTMFRIPWKHAGKQDFREDQDAAFFKAWAIFKGKYKEGDTGGPAVWKTRLRCALNK

SEQ ID NO: 3 - protein, 89 aa, Homo sapiens
MALHPRRVRLKPWLVAQVDSGLYPGLIWLHRDSKRFQIPWKHATRHSPQQEEENTIFKAWAVETGKYQEGVDDPDPAKWKAQLRCALNK

SEQ ID NO: 4 - protein, 89 aa, Homo sapiens; identical to SEQ ID NO: 3

SEQ ID NO: 5 - protein, 96 aa, Homo sapiens
MNQSIPVAPTPPRRVRLKPWLVAQVNSCQYPGLQWVNGEKKLFCIPWRHATRHGPSQDGDNTIFKAWAKETGKYTEGVDEADPAKWKANLRCALNK

SEQ ID NO: 6 - protein, 87 aa, Homo sapiens
MPITRMRMRPWLEMQINSNQIPGLIWINKEEMIFQIPWKHAAKHGWDINKDACLFRSWAIHTGRYKAGEKEPDPKTWKANFRCAMNS

SEQ ID NO: 7 - protein, 87 aa, Homo sapiens
MPVERMRMRPWLEEQINSNTIPGLKWLNKEKKIFQIPWMHAARHGWDVEKDAPLFRNWAIHTGKHQPGVDKPDPKTWKANFRCAMNS

SEQ ID NO: 8 - protein, 97 aa, Homo sapiens
MSNIPSPQTCEKTSNKRKPRQICCDSNKRQIYPHDTSISTEAEVSEIKFRFGPELNFYKNTAARLQSFNHNDQFYNPRFRPHIRTNRKKSESTNYTD

SEQ ID NO: 9 - DNA, 15 bp, Artificial
  Synthetic oligomer
gggaaaccgaaactg

SEQ ID NO: 10 - DNA, 12 bp, Artificial
  Synthetic oligonucleotides
gtccaattggtc